(19) United States
(12) Patent Application Publication
(10) Pub. No.: US 2024/0238251 A9
(43) Pub. Date: Jul. 18, 2024

(54) A Composition Comprising Heterocyclic Compounds, Preparation Method and Application Thereof

(71) Applicants: SHANGHAI YASHENG PHARMACEUTICAL TECHNOLOGY CO., LTD., Shanghai (CN); ASCENTAGE PHARMA (SUZHOU) CO., LTD., Suzhou (CN); ASCENTAGE PHARMA GROUP CORP LIMITED, Hong Kong (CN)

(72) Inventors: Yanqiong LIN, Shanghai (CN); Hualiang XU, Shanghai (CN); Hong-tao GUO, Shanghai (CN)

(21) Appl. No.: 18/250,861

(22) PCT Filed: Nov. 3, 2021
(86) PCT No.: PCT/CN2021/128402

§ 371 Date: Apr. 27, 2023

(30) Foreign Application Priority Data
Nov. 3, 2020 (CN) 202011206371.7

(51) Int. Cl. A61K 31/407; A61K 9/20; A61K 47/38

(52) U.S. Cl. CPC A61K 31/407; A61K 9/2036; A61K 9/2054; A61K 47/38

(57) ABSTRACT

Provided herein is a solid dispersion, which comprises a carrier and an active ingredient. The active ingredient is one or more of the compound shown in formula (I) (APG-115), its pharmaceutically acceptable salt, its crystal form and its hydrate. The solid dispersion can improve the dissolution of the active ingredient APG-115. The dissolution of APG-115 of some solid dispersions can reach more than 90%, and has good stability. It can improve the dissolution and dissolution of drugs in gastrointestinal fluid, so as to improve the oral bioavailability. The solid dispersion shows high plasma exposure in animals, that is, higher drug peak concentration and higher area under blood concentration curve.

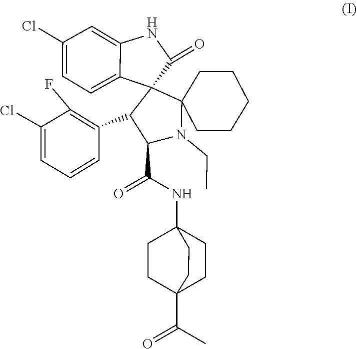

This application claims priority to Chinese patent application CN202011206371.7 filed on Nov. 3, 2020. The contents of which are incorporated herein by its entirety.

### TECHNICAL FIELD

The invention relates to a composition comprising heterocyclic compounds, a preparation method and application thereof.

### BACKGROUND ART

MDM2 is an oncogene that regulates cell growth. MDM2 can combine with p53 protein to form MDM2-p53 negative feedback loop and exert p53 dependent activity. MDM2 is the main inhibitor of p53. It inhibits the function of p53 through a variety of mechanisms, which are mediated by their interactions. Small molecule inhibitors that block the interaction of MDM2-p53 may achieve the purpose of treating human cancer by restoring the tumor inhibitory function of wild-type p53.

APG-115 is an oral effective and highly selective small molecule inhibitor targeting MDM2-p53 protein interaction, and has been clinically studied.

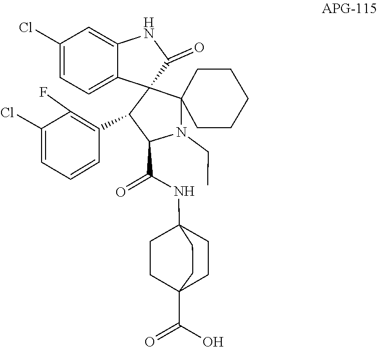 [SMILES: CCN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2]

### SUMMARY OF THE INVENTION

The invention provides a solid dispersion, which comprises a carrier and an active ingredient; The active ingredient is one or more of a compound as shown in formula (I), its pharmaceutically acceptable salt, its crystal form and its hydrate;

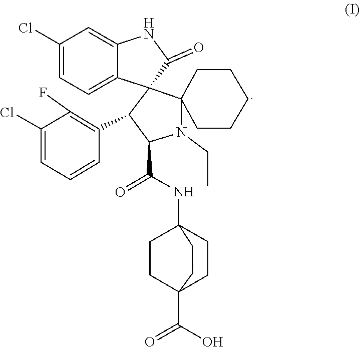 [SMILES: CCN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2]

In an embodiment of the invention, the carrier is selected from one or more of homopolymer and copolymer cellulose ester of N-vinyl lactam, pH dependent cellulose derivative, non-ionic water-soluble cellulose ether, cellulose ether, high molecular weight polycyclic oxide, N-vinyl amide polymer, polyacrylate, polymethacrylate, polyacrylamide, vinyl acetate polymer, polyethylene glycol, polyvinyl caprolactam/polyvinyl acetate graft copolymer, oligosaccharide and polysaccharide, and can also be one or more of povidone, covidone, hypromellose acetate succinate, polyethylene glycol/polyvinyl caprolactam/polyvinyl acetate graft copolymer, and can also be one or more of hypromellose acetate succinate, hydroxypropyl cellulose, povidone and acrylic resin.

In an embodiment of the invention, the hypromellose acetate succinate includes but is not limited to one or more of HPMCAS 126G, HPMCAS 716G and HPMCAS 912G.

In an embodiment of the invention, the Hydroxypropyl cellulose includes but is not limited to one or more of HPC EXF, HPC LF, HPC JF and HPC GF, and can also be HPC EXF.

In an embodiment of the invention, the povidone includes but is not limited to one or more of PVP VA 64, PVP K29/32, PVP S-630, PVP K25, PVP K-90, PVP C-15 and PVP C-30, or one or more of PVP VA64, PVP K29/32 and PVP S-630.

In an embodiment of the invention, the acrylic resin includes but is not limited to Eudragit L100, Eudragit S100, Eudragit L100-55, Eudragit RLPO and Eudragit RSPO, and can also be one or more of Eudragit L100, Eudragit S100 and Eudragit L100-55.

In an embodiment of the invention, the pharmaceutically acceptable salt of the compound shown in formula (I) can be hydrochloride, hydrobromate, hydroiodate, sulfate, hydrogen sulfate, 2-hydroxyethanesulfonate, phosphate, hydrogen phosphate, acetate, adipate, alginate, lysine, arginine, histidine, aspartate, benzoate, bisulfate Butyrate, camphorate, camphorsulfonate, digluconate, glycerophosphate, hemisulfate, heptanate, caproate, formate, succinate, fumaric acid, maleate, ascorbate, hydroxyethyl sulfonate, salicylate, methanesulfonate, mesitylene sulfonate, naphthalene sulfonate, nicotinate, 2-naphthalene sulfonate, oxalate Dihydroxynaphthenate, pectinate, persulfate, 3-phenylpropionate, picrate, tervalerate, propionate, trichloroacetate, trifluoroacetate, phosphate, glutamate, bicarbonate, p-toluenesulfonate, undecanoate, lactate, citrate, tartrate, gluconate, methanesulfonate, ethyldisulfonate, benzene sulfonate, 1-tartrate maleate, sodium salt, potassium salt, choline salt, aminobutanol salt, calcium salt or p-toluenesulfonate, or phosphate, sulfate, 1-tartrate, hydrochloride, maleate, hydrobromate, methanesulfonate, lysine, arginine, histidine salt, sodium salt, potassium salt, choline salt, aminobutanol salt or calcium salt.

In an embodiment of the invention, the hydrate of the compound shown in formula (I) can be hemihydrate, monohydrate, dihydrate, trihydrate, tetrahydrate, pentahydrate, hexahydrate, heptahydrate, octahydrate, nonahydrate, decahydrate, undecahydrate or dodecahydrate.

The carrier of the invention, such as hypromellose acetate succinate (HPMCAS), is commercially available, such as HPMCAS 126G, HPMCAS 716G, HPMCAS 912G, HPMCAS HG, HPMCAS LG and HPMCAS MG, or HPMCAS is selected from acetyl substitution degree (%) 5.0-9.0, succinyl substitution degree (%) 14.0-18.0 (for example, HPMCAS 716G or HPMCAS LG), or HPMCAS is selected from acetyl substitution degree (%) 7.0-11.0 and succinyl substitution degree (%) 10.0-14.0 (for example, HPMCAS 912G or HPMCAS MG), or HPMCAS is selected from acetyl substitution degree (%) 10.0-14.0 and succinyl substitution degree (%) 4.0-8.0 (for example, HPMCAS 126G or HPMCAS HG).

Other polymers such as povidone (PVP), hydroxypropyl cellulose (HPC) and acrylic resin (AC) is also commercially available. For example, povidone (PVP) comes from PVP K12, PVP K15, PVP K 17, PVP K 30, PVP K 60, PVP C-12, PVP C-17, PVP VA64, PVP K29/32, PVP S-630, PVP K25, PVP K-90, PVP C-15, PVP C-30, PVP VA64, PVP K29/32 and PVP S-630. For example, hydroxypropyl cellulose (HPC) comes from HPC EXF, HPC LF, HPC JF, HPC GF, HPC EXF, HPC H (including HF and HXF), HPC M (including MF and MXF), HPC G (including GF and GXF), HPC J (including JF and JXF), HPC L (including LF and LXF), HPC E (including EF and EXF), HPC L, HPC M, HPC H, HPC SL and HPC SSL. For example, acrylic resin (AC) is selected from E100, EPO, 1100-55, L100, S100, RL100, RLPO, RL30D, RS100, RSPO, RS30D, NE30D and RD100.

In an embodiment of the invention, the carrier can be one or more of HPMCAS 912G (or HPMCAS MG), HPC Klucel EXF, Eudragit S100 and Eudragit L100-55. The obtained dispersion has no obvious endothermic and exothermic peaks under DSC scanning, and the obtained dispersion is amorphous dispersion.

In an embodiment of the invention, the carrier can be one or more of HPMCAS 912G (or HPMCAS MG), HPMCAS 126G (or HPMCAS HG), HPC Klucel EXF, PVP VA64, PVP K29/32 and Eudragit L100-55. The obtained dispersion has good stability.

In an embodiment of the invention, the carrier can be one or more of HPMCAS 716G (or HPMCAS LG), HPMCAS 912G (or HPMCAS MG) and PVP VA64. The solubility of the obtained dispersion is significantly improved (the equilibrium solubility is greater than 0.1 mg/ml).

In an embodiment of the invention, the carrier can be one or more of HPMCAS 126G (or HPMCAS HG), HPMCAS 716G (or HPMCAS LG), HPMCAS 912G (or HPMCAS MG), Eudragit L100 and Eudragit S100. The drug dissolution of the obtained dispersion was complete (the drug dissolution in 10 min is greater than 50%, and the drug dissolution in 60 min is greater than 97%).

In an embodiment of the invention, the carrier can be HPMCAS912G (or HPMCAS MG).

In an embodiment of the invention, the active ingredient can be a compound as shown in formula (I).

In an embodiment of the invention, in the solid dispersion, the mass ratio of the active ingredient to the carrier can be 1:1.2˜1:8 or 1:2˜1:4 (for example 1:2). When the mass ratio of active ingredient to carrier is in the range of 1:1.2˜1:8, the solid dispersion has good effects in at least one aspect, such as equilibrium solubility, stability or drug dissolution. For example, the dissolution can reach more than 90%, for example, the equilibrium solubility is greater than 0.1 mg/ml. When the mass ratio of active ingredient to carrier is in the range of 1:2˜1:4, the equilibrium solubility, stability or drug dissolution of solid dispersion have better effects (for example, the dissolution can be greater than 98%, and the content has no significant change with related substances after 30 days at 75% RH or 60° C.).

In an embodiment of the invention, by mass of 1 part of the active ingredient, the solid dispersion may include 1 part of the active ingredient and 1.2˜8 parts of the carrier.

In an embodiment of the invention, by mass of 1 part of the active ingredient, the solid dispersion may include 1 part of the active ingredient and 2˜4 parts of the carrier.

In an embodiment of the invention, by mass of 1 part of the active ingredient, the solid dispersion may include 1 part of the active ingredient and 1.2˜8 parts of hypromellose acetate succinate.

In an embodiment of the invention, by mass of 1 part of the active ingredient, the solid dispersion may include 1 part of the active ingredient and 2˜4 parts of hypromellose acetate succinate.

In an embodiment of the invention, by mass of 1 part of the active ingredient, the solid dispersion may include 1 part of the active ingredient and 2˜4 parts of HPMCAS 912G (or HPMCAS MG).

In an embodiment of the invention, by mass of 1 part of the active ingredient, the solid dispersion may include 1 part of the active ingredient and 2˜4 parts of HPMCAS 126G (or HPMCAS HG).

In an embodiment of the invention, by mass of 1 part of the active ingredient, the solid dispersion may include 1 part of the active ingredient and 2˜4 parts of HPMCAS 716G (or HPMCAS LG).

In an embodiment of the invention, by mass of 1 part of the active ingredient, the solid dispersion comprises 1 part of the active ingredient and 2 parts of hypromellose acetate succinate.

In an embodiment of the invention, by mass of 1 part of the active ingredient, the solid dispersion may include 1 part of the active ingredient and 2 parts of HPMCAS 912G (or HPMCAS MG).

In an embodiment of the invention, by mass of 1 part of the active ingredient, the solid dispersion may include 1 part of the active ingredient and 2 parts of HPMCAS 126G (or HPMCAS HG).

In an embodiment of the invention, by mass of 1 part of the active ingredient, the solid dispersion may include 1 part of the active ingredient and 2 parts of HPMCAS 716G (or HPMCAS LG).

In an embodiment of the invention, by mass of 1 part of the active ingredient, the solid dispersion may include 1 part of the active ingredient and 1.2˜8 parts of hydroxypropyl cellulose.

In an embodiment of the invention, by mass of 1 part of the active ingredient, the solid dispersion may include 1 part of the active ingredient and 2˜4 parts of hydroxypropyl cellulose.

In an embodiment of the invention, by mass of 1 part of the active ingredient, the solid dispersion may include 1 part of the active ingredient and 2˜4 parts of HPC EXF.

In an embodiment of the invention, by mass of 1 part of the active ingredient, the solid dispersion comprises 1 part of the active ingredient and 2 parts of hydroxypropyl cellulose.

In an embodiment of the invention, by mass of 1 part of the active ingredient, the solid dispersion may include 1 part of the active ingredient and 2 parts of HPC EXF.

In an embodiment of the invention, by mass of 1 part of the active ingredient, the solid dispersion may include 1 part of the active ingredient and 1.2˜8 parts of povidone.

In an embodiment of the invention, by mass of 1 part of the active ingredient, the solid dispersion may include 1 part of the active ingredient and 2˜4 parts of povidone.

In an embodiment of the invention, by mass of 1 part of the active ingredient, the solid dispersion may include 1 part of the active ingredient and 2˜4 parts of PVP VA64.

In an embodiment of the invention, by mass of 1 part of the active ingredient, the solid dispersion may include 1 part of the active ingredient and 2˜4 parts of PVP K29/32.

In an embodiment of the invention, by mass of 1 part of the active ingredient, the solid dispersion may include 1 part of the active ingredient and 2˜4 parts of PVP S-630.

In an embodiment of the invention, by mass of 1 part of the active ingredient, the solid dispersion comprises 1 part of the active ingredient and 2 parts of povidone.

In an embodiment of the invention, by mass of 1 part of the active ingredient, the solid dispersion may include 1 part of the active ingredient and 2 parts of PVP VA64.

In an embodiment of the invention, by mass of 1 part of the active ingredient, the solid dispersion may include 1 part of the active ingredient and 2 parts of PVP K29/32.

In an embodiment of the invention, by mass of 1 part of the active ingredient, the solid dispersion may include 1 part of the active ingredient and 2 parts of PVP S-630.

In an embodiment of the invention, by mass of 1 part of the active ingredient, the solid dispersion may include 1 part of the active ingredient and 1.2˜8 parts of acrylic resin.

In an embodiment of the invention, by mass of 1 part of the active ingredient, the solid dispersion may include 1 part of the active ingredient and 2˜4 parts of acrylic resin.

In an embodiment of the invention, by mass of 1 part of the active ingredient, the solid dispersion may include 1 part of the active ingredient and 2˜4 parts of Eudragit L100.

In an embodiment of the invention, by mass of 1 part of the active ingredient, the solid dispersion may include 1 part of the active ingredient and 2˜4 parts of Eudragit S100.

In an embodiment of the invention, by mass of 1 part of the active ingredient, the solid dispersion may include 1 part of the active ingredient and 2˜4 parts of Eudragit L100-55.

In an embodiment of the invention, by mass of 1 part of the active ingredient, the solid dispersion comprises 1 part of the active ingredient and 2 parts of acrylic resin.

In an embodiment of the invention, by mass of 1 part of the active ingredient, the solid dispersion may include 1 part of the active ingredient and 2 parts of Eudragit L100.

In an embodiment of the invention, by mass of 1 part of the active ingredient, the solid dispersion may include 1 part of the active ingredient and 2 parts of Eudragit S100.

In an embodiment of the invention, by mass of 1 part of the active ingredient, the solid dispersion may include 1 part of the active ingredient and 2 parts of Eudragit L100-55.

In an embodiment of the invention, the solid dispersion further comprises an antisticking agent. The antisticking agent may include one or more of colloidal silica, talc powder, starch, D-leucine, L-leucine, sodium lauryl sulfate and metal stearate; For example, the antisticking agent can be colloidal silica. The mass ratio of the antisticking agent to the active ingredient can be 0.05:1˜0.08:1 (for example 0.064:1).

In an embodiment of the invention, by mass of 1 part of the active ingredient, the solid dispersion may include 1 part of the active ingredient, 1.2˜8 parts of the carrier and 0.05˜0.08 parts of the antisticking agent.

In an embodiment of the invention, by mass of 1 part of the active ingredient, the solid dispersion may include 1 part of the active ingredient, 1.2˜8 parts of hypromellose acetate succinate and 0.05˜0.08 parts of colloidal silica.

In an embodiment of the invention, by mass of 1 part of the active ingredient, the solid dispersion may include 1 part of the active ingredient, 1.2˜8 parts of HPMCAS 912G (or HPMCAS MG) and 0.05˜0.08 parts of colloidal silica.

In an embodiment of the invention, by mass of 1 part of the active ingredient, the solid dispersion may include 1 part of the active ingredient, 2˜4 parts of hypromellose acetate succinate and 0.06˜0.07 parts of colloidal silica.

In an embodiment of the invention, by mass of 1 part of the active ingredient, the solid dispersion may include 1 part of the active ingredient, 2˜4 parts of HPMCAS 912G (or HPMCAS MG) and 0.06˜0.07 parts of colloidal silica.

In an embodiment of the invention, by mass of 1 part of the active ingredient, the solid dispersion may include 1 part of the active ingredient, 2 parts of hypromellose acetate succinate and 0.064 parts of colloidal silica.

In an embodiment of the invention, by mass of 1 part of the active ingredient, the solid dispersion may include 1 part of the active ingredient, 2 parts of HPMCAS 912G (or HPMCAS MG) and 0.064 parts of colloidal silica.

In an embodiment of the invention, the solid dispersion can be composed of the carriers and the active ingredient.

In an embodiment of the invention, the solid dispersion is composed of the carriers, the active ingredient and the antisticking agents.

In an embodiment of the invention, the solid dispersion is an amorphous solid dispersion.

The invention also provides a method for preparing a solid dispersion, which comprises the following steps: the carrier and the active ingredient are mixed with a solvent to obtain a mixed liquid, and then the mixed liquid is dried; Optionally, the mixture is mixed with the antisticking agent before drying.

In an embodiment of the invention, the mass volume ratio of the active ingredient to the solvent can be 5:1˜30:1 mg/ml, or 15:1˜25:1 mg/ml (for example 19.5:1 mg/ml). When the mass volume ratio of the active ingredient to the solvent is in the range of 5:1˜30:1 mg/ml, the stability of the active ingredient is good, and the solvent residue of the prepared solid dispersion is low (for example, it can be lower than the solvent limit).

In an embodiment of the invention, the solvent can be one or more of alcohol solvent, water, ester solvent, ketone solvent, halogenated hydrocarbon solvent, nitrile solvent and ether solvent, ester solvent and/or ether solvent, or ester solvent. The alcohol solvent can be ethanol. The ester solvent can be methyl acetate. The ether solvent can be tetrahydrofuran. The ketone solvent can be acetone. The halogenated hydrocarbon solvent can be dichloromethane. The nitrile solvent can be acetonitrile.

In an embodiment of the invention, the drying may include a first drying and a second drying; The first drying can be spray drying or fluidized bed boiling drying. The second drying can be vacuum decompression drying or electric blast drying, such as vacuum decompression drying.

In an embodiment of the invention, the end point of drying is when the solvent residue in the solid dispersion is within the limit.

In an embodiment of the invention, the drying time can be 4˜16 hours. The first drying time can be 2 to 8 hours (for example, 5 hours). The second drying time can be 2 to 8 hours (for example, 2 hours).

The inventor found that the antisticking agent could improve the electrostatic adhesion of powders during spray drying. When the mass ratio of anti sticking agent to active ingredient is in the range of 0.05:1˜0.08:1, the effect of improving powder electrostatic adhesion in the drying process is better.

The invention also provides a solid dispersion prepared by the preparation method of the solid dispersion.

The invention also provides a pharmaceutical composition comprising the solid dispersion and excipient.

The excipients for preparing the above pharmaceutical composition may include encapsulation materials or preparation additives, for example, one or more of absorption enhancers, antioxidants, dry antisticking agents, buffers, coating materials, coloring agents, diluents, disintegrating agents, emulsifiers, flavor agents, humectants, lubricants, antisticking agents, glidants, preservatives, solubilizers, correctives and releasing agents. Excipients can have two or more functions in drug combinations.

The invention also provides a pharmaceutical composition comprising the solid dispersion, diluent, disintegrating agent and lubricant.

In an embodiment of the invention, the diluent may include one or more of cellulose (for example, one or more of powdered cellulose, microcrystalline cellulose, silicified microcrystalline cellulose α- and amorphous cellulose, cellulose acetate, etc.), lactose (for example anhydrous lactose and/or lactose monohydrate), lactoitol, maltitol, mannitol, sorbitol, xylitol, glucose (for example anhydrous glucose and/or glucose monohydrate), fructose, sucrose and sucrose based diluents (for example, one or more of compressible sugar, powdered sugar, sugar pills, etc.), maltose, inositol, hydrolyzed grain solids, starch (for example, one or more of corn starch, wheat starch, rice starch, potato starch, cassava starch, etc.), starch components (for example, one or more of amylose and glucose binders, and modified starch or processed starch, such as pregelatinized starch), dextrin, calcium salt (for example, one or more of calcium carbonate, calcium phosphate, calcium sulfate and calcium lactate), magnesium salt (for example, magnesium carbonate and/or magnesium oxide), bentonite, kaolin and sodium chloride.

In an embodiment of the invention, the diluent can be one or more of microcrystalline cellulose, silicified microcrystalline cellulose, calcium phosphate, pregelatinized starch, lactose, mannitol and anhydrous calcium hydrogen phosphate, one or more of microcrystalline cellulose, silicified microcrystalline cellulose, pregelatinized starch, calcium phosphate and anhydrous calcium hydrogen phosphate, or one or more of microcrystalline cellulose, pregelatinized starch and calcium phosphate, or microcrystalline cellulose and anhydrous calcium hydrogen phosphate. The microcrystalline cellulose can be PH 102 and/or KG 802. When the diluent is microcrystalline cellulose and anhydrous calcium hydrogen phosphate, the mass ratio of microcrystalline cellulose to anhydrous calcium hydrogen phosphate can be 0.1:1˜10:1, or 0.5:1˜2:1 (for example, 1:1).

In an embodiment of the invention, the disintegrating agent may include starch (for example, pregelatinized starch and/or sodium starch glycolate), clay, magnesium aluminum silicate and cellulose based disintegrating agent (for example, powdered cellulose, microcrystalline cellulose, methylcellulose, low-grade alternative hydroxypropyl cellulose, carboxymethylcellulose, carboxymethylcellulose calcium, carboxymethylcellulose sodium and croscarmellose sodium), alginate, Povidone, crospovidone, potassium polaclin, gum (for example, one or more of agar, guar gum, locust bean gum, karaya gum, pectin and tragacanth gum, etc.) and colloidal silica.

In an embodiment of the invention, the disintegrating agent can be croscarmellose sodium and/or crospovidone.

The lubricants reduce the friction between materials and equipment of solid preparations in preparation molding, such as reducing the friction between tablet pressing mixture and tablet pressing equipment in tablet pressing process. In an embodiment of the present invention, the lubricant may include one or more of glyceryl behenate, stearic acid and its salts (for example, one or more of magnesium stearate, calcium stearate, sodium stearate, etc.), hydrogenated vegetable oil, glyceryl palmitate stearate, talc powder, wax, sodium benzoate, sodium acetate, sodium fumarate, sodium stearate fumarate, PEG (for example, PEG4000 and/or PEG6000), poloxamer, polyvinyl alcohol, sodium oleate, sodium lauryl sulfate and magnesium lauryl sulfate.

In an embodiment of the invention, the lubricant can be one or more of magnesium stearate, polyethylene glycol and magnesium lauryl sulfate, or magnesium stearate.

Other excipients, such as dry antisticking agents, buffers, stabilizers, solubilizers, antioxidants, coloring agents, correctives, etc., are known in the pharmaceutical field and can be used in the composition of the present invention. The tablets can be uncoated or may comprise tablets coated, for example, with a non-functional membrane or release modified or enteric coating. The capsule may have a hard or soft shell comprising optionally one or more plasticizers such as gelatin (in the form of hard gelatin capsule or soft elastic gelatin capsule), starch, carrageenan and/or hydroxypropyl cellulose.

In an embodiment of the invention, the mass ratio of the diluent to the solid dispersion can be 0.2:1˜8:1, 0.5:1˜8:1, 0.8:1˜2:1 or 0.5:1˜1:1 (for example 1.15:1, 0.76:1, 1.36:1). When the mass ratio of diluent to solid dispersion is in the range of 0.2:1˜8:1, film side of the plain tablet made of pharmaceutical composition is white and free of spots.

In an embodiment of the invention, the mass ratio of the disintegrating agent to the solid dispersion can be 0.03:1˜0.3:1, 0.1:1˜0.2:1, 0.05:1˜0.2:1 or 0.05:1˜0.15:1 (for example 0.15:1, 0.13:1). When the mass ratio of disintegrating agent to solid dispersion is in the range of 0.03:1˜0.3:1, the tablets made of pharmaceutical composition have good disintegration and dissolution effects (for example, the disintegration time can be between 0.5 min and 7.5 min, and the dissolution can be more than 90%).

In an embodiment of the invention, the mass ratio of the lubricant to the solid dispersion can be 0.005:1˜0.2:1, 0.01:1˜0.2:1, 0.02:1˜0.04:1 or 0.01:1˜0.02:1 (for example 0.042:1, 0.026:1). When the mass ratio of lubricant to solid dispersion is in the range of 0.005:1˜0.2:1, the powder made of pharmaceutical composition has better fluidity.

In an embodiment of the invention, by mass of 1 part of the solid dispersion, the pharmaceutical composition may include 1 part of the solid dispersion, 0.2˜8 parts of the diluent, 0.03˜0.3 parts of the disintegrating agent and 0.005˜0.2 parts of the lubricant.

In an embodiment of the invention, by mass of 1 part of the solid dispersion, the pharmaceutical composition may include 1 part of the solid dispersion, 0.5˜1 part of silicified microcrystalline cellulose, 0.1˜0.2 part of Crospovidone and 0.01˜0.02 part of magnesium stearate.

In an embodiment of the invention, by mass of 1 part of the solid dispersion, the pharmaceutical composition may include 1 part of the solid dispersion, 0.65˜0.85 parts of silicified microcrystalline cellulose, 0.13˜0.17 parts of Crospovidone and 0.012˜0.016 parts of magnesium stearate.

In an embodiment of the invention, by mass of 1 part of the solid dispersion, the pharmaceutical composition may include 1 part of the solid dispersion, 0.76 part of silicified microcrystalline cellulose, 0.15 part of Crospovidone and 0.014 part of magnesium stearate.

In an embodiment of the invention, the pharmaceutical composition is composed of the solid dispersion, the diluents, the disintegrations and the lubricants.

In an embodiment of the invention, the pharmaceutical composition may also include antisticking agents and glidants.

The antisticking agents can reduce the adhesion to the equipment surface during the preparation of the preparation. In an embodiment of the invention, the antisticking agent may include one or more of talc powder, colloidal silica, starch, D-leucine, L-leucine, sodium lauryl sulfate, metal stearate, etc.

In an embodiment of the invention, the antisticking agent can be one or more of colloidal silica, talc powder and calcium chloride, or colloidal silica.

Glidants improve the flow properties and reduce static electricity in the tablet mixture. In an embodiment of the invention, the glidant may include one or more of colloidal silica, starch, powdered cellulose, sodium lauryl sulfate, magnesium trisilicate, metal stearate, etc.

In an embodiment of the invention, the glidant can be colloidal silica and/or talc powder, or colloidal silica.

In an embodiment of the invention, in the pharmaceutical composition, the mass ratio of the total mass of the antisticking agent and glidant to the solid dispersion can be 0.02:1˜0.3:1, 0.05:1˜0.1:1 or 0.1:1˜0.2:1 (for example 0.08:1, 0.13:1). When the mass ratio of anti sticking agent and glidant to the mass ratio of solid dispersion is in the range of 0.02:1˜0.3:1, it has a better effect on preventing film on the surface of punch and reducing the sticking impact of tablet during tablet pressing.

In an embodiment of the invention, by mass of 1 part of the solid dispersion, the pharmaceutical composition may include 1 part of solid dispersion, 0.2˜8 parts of diluent, 0.02 parts (the antisticking agent and glidant), 0.03˜0.3 parts of disintegrating agent and 0.005˜0.2 parts of lubricant.

In an embodiment of the invention, by mass of 1 part of the solid dispersion, the pharmaceutical composition may include 1 part of the solid dispersion, 0.8˜2 parts of microcrystalline cellulose PH 102, 0.05˜0.2 parts of croscarmellose sodium, 0.05˜0.1 parts of colloidal silica and 0.02˜0.04 parts of magnesium stearate.

In an embodiment of the invention, by mass of 1 part of the solid dispersion, the pharmaceutical composition may include 1 part of the solid dispersion, 1.26˜1.46 parts of microcrystalline cellulose PH 102, 0.11˜0.15 parts of croscarmellose sodium, 0.06˜0.1 parts of colloidal silica and 0.02˜0.04 parts of magnesium stearate.

In an embodiment of the invention, by mass of 1 part of the solid dispersion, the pharmaceutical composition may include 1 part of the solid dispersion, 0.53˜1.32 parts of microcrystalline cellulose PH 102, 0.27˜0.68 parts of pregelatinized starch, 0.05˜0.2 parts of croscarmellose sodium, 0.05˜0.1 parts of colloidal silica and 0.02˜0.04 parts of magnesium stearate.

In an embodiment of the invention, by mass of 1 part of the solid dispersion, the pharmaceutical composition may include 1 part of the solid dispersion, 0.81˜1.01 part of microcrystalline cellulose PH 102, 0.35˜0.55 part of pregelatinized starch, 0.11˜0.15 part of croscarmellose sodium, 0.06˜0.1 part of colloidal silica and 0.02˜0.04 part of magnesium stearate.

In an embodiment of the invention, by mass of 1 part of the solid dispersion, the pharmaceutical composition may include 1 part of the solid dispersion, 0.4˜1 part of microcrystalline cellulose PH 102, 0.4˜1 part of calcium phosphate, 0.05˜0.2 part of croscarmellose sodium, 0.05˜0.1 part of colloidal silica and 0.02˜0.04 part of magnesium stearate.

In an embodiment of the invention, by mass of 1 part of the solid dispersion, the pharmaceutical composition may include 1 part of the solid dispersion, 0.58˜0.78 parts of microcrystalline cellulose PH 102, 0.58˜0.78 parts of calcium phosphate, 0.11˜0.15 parts of croscarmellose sodium, 0.07˜0.09 parts of colloidal silica and 0.02˜0.04 parts of magnesium stearate.

In an embodiment of the invention, by mass of 1 part of the solid dispersion, the pharmaceutical composition may include 1 part of the solid dispersion, 0.4˜1 part of microcrystalline cellulose KG 802, 0.4˜1 part of anhydrous calcium phosphate, 0.05˜0.2 part of croscarmellose sodium, 0.05˜0.1 part of colloidal silica and 0.02˜0.04 part of magnesium stearate.

In an embodiment of the invention, by mass of 1 part of the solid dispersion, the pharmaceutical composition may include 1 part of the solid dispersion, 0.61˜0.81 parts of microcrystalline cellulose KG 802, 0.61˜0.81 parts of anhydrous calcium phosphate, 0.068˜0.088 parts of croscarmellose sodium, 0.05˜0.054 parts of colloidal silica and 0.03˜0.04 parts of magnesium stearate.

In an embodiment of the invention, by mass of 1 part of the solid dispersion, the pharmaceutical composition may include 1 part of the solid dispersion, 1.36 parts of microcrystalline cellulose PH 102, 0.13 parts of croscarmellose sodium, 0.08 parts of colloidal silica and 0.03 parts of magnesium stearate.

In an embodiment of the invention, by mass of 1 part of the solid dispersion, the pharmaceutical composition may include 1 part of the solid dispersion, 0.91 part of microcrystalline cellulose PH 102, 0.45 part of pregelatinized starch, 0.13 part of croscarmellose sodium, 0.08 part of colloidal silica and 0.03 part of magnesium stearate.

In an embodiment of the invention, by mass of 1 part of the solid dispersion, the pharmaceutical composition may include 1 part of the solid dispersion, 0.68 part of microcrystalline cellulose PH 102, 0.68 part of calcium phosphate, 0.13 part of croscarmellose sodium, 0.08 part of colloidal silica and 0.03 part of magnesium stearate.

In an embodiment of the invention, by mass of 1 part of the solid dispersion, the pharmaceutical composition may include 1 part of the solid dispersion, 0.71 part of microcrystalline cellulose KG 802, 0.71 part of anhydrous calcium phosphate, 0.078 part of croscarmellose sodium, 0.052 part of colloidal silica and 0.04 part of magnesium stearate.

In an embodiment of the invention, the pharmaceutical composition is composed of the solid dispersion, the diluent, the disintegrating agent, the antisticking agent, the glidant and the lubricant.

In an embodiment of the invention, the pharmaceutical composition may also include a dry antisticking agent.

In an embodiment of the invention, the dry antisticking agent may include one or more of gum Arabic, tragacanth gum, glucose, polyglucose, starch (for example, pregelatinized starch), gelatin, modified cellulose (for example, one or more of methylcellulose, sodium carboxymethylcellulose, hydroxypropyl methyl cellulose, hydroxypropyl cellulose, hydroxyethyl cellulose and ethyl cellulose) Dextrin (for example, maltodextrin), zein, alginic acid and alginate (for example, sodium alginate), magnesium aluminum silicate, bentonite, polyethylene glycol, polyethylene oxide, guar gum, polysaccharide acid, polyvinylpyrrolidone, polyacrylic acid (for example, carbomer), polymethacrylate, etc.

In an embodiment of the invention, in the pharmaceutical composition, the dry antisticking agent can be hydroxypropyl cellulose and/or hydroxypropyl methyl cellulose, or hydroxypropyl cellulose.

In an embodiment of the invention, in the pharmaceutical composition, the mass ratio of the dry antisticking agent to the solid dispersion can be 0.02:1˜0.5:1, or 0.1:1˜0.3:1 (for example 0.13:1). When the mass ratio of dry antisticking agent to solid dispersion is in the range of 0.02:1˜0.5:1, the plain tablets made of pharmaceutical composition have higher hardness and better friability; When the mass ratio is in the range of 0.1:1˜0.3:1, the plain tablets made of the pharmaceutical composition have higher hardness and better friability (for example, the hardness can be between 60 and 170N and the friability can be between 0.07%-0.55% (200 rpm).

In an embodiment of the invention, by mass of 1 part of the solid dispersion, the pharmaceutical composition may include 1 part of the solid dispersion, 0.2˜8 parts of the diluent, 0.02˜0.5 parts of the dry anti sticking agent, 0.02˜0.3 parts (the anti sticking agent and glidant), 0.03˜0.3 parts of the disintegrating agent and 0.005˜0.2 parts of the lubricant.

In an embodiment of the invention, by mass of 1 part of the solid dispersion, the pharmaceutical composition may include 1 part of the solid dispersion, 0.25˜4 parts of microcrystalline cellulose KG 802, 0.25˜4 parts of calcium phosphate, 0.03˜0.3 parts of croscarmellose sodium, 0.02˜0.5 parts of hydroxypropyl cellulose, 0.02˜0.3 parts of colloidal silica and 0.02˜0.3 parts of magnesium stearate.

In an embodiment of the invention, by mass of 1 part of the solid dispersion, the pharmaceutical composition may include 1 part of the solid dispersion, 0.5˜0.7 parts of microcrystalline cellulose KG 802, 0.5˜0.7 parts of calcium phosphate, 0.11˜0.15 parts of croscarmellose sodium, 0.14˜0.18 parts of hydroxypropyl cellulose, 0.04˜0.08 parts of colloidal silica and 0.02˜0.04 parts of magnesium stearate.

In an embodiment of the invention, by mass of 1 part of the solid dispersion, the pharmaceutical composition may include 1 part of the solid dispersion, 0.25˜4 parts of microcrystalline cellulose KG 802, 0.25˜4 parts of anhydrous calcium phosphate, 0.03˜0.3 parts of croscarmellose sodium, 0.02˜0.5 parts of hydroxypropyl cellulose, 0.02˜0.3 parts of colloidal silica and 0.02˜0.3 parts of magnesium stearate.

In an embodiment of the invention, by mass of 1 part of the solid dispersion, the pharmaceutical composition may include 1 part of the solid dispersion, 0.55˜0.75 parts of microcrystalline cellulose KG 802, 0.55˜0.75 parts of anhydrous calcium phosphate, 0.07˜0.15 parts of croscarmellose sodium, 0.07˜0.18 parts of hydroxypropyl cellulose, 0.04˜0.15 parts of colloidal silica and 0.02˜0.06 parts of magnesium stearate.

In an embodiment of the invention, by mass of 1 part of the solid dispersion, the pharmaceutical composition may include 1 part of the solid dispersion, 0.61 part of microcrystalline cellulose KG 802, 0.61 part of calcium phosphate, 0.13 part of croscarmellose sodium, 0.16 part of hydroxypropyl cellulose, 0.06 part of colloidal silica and 0.03 part of magnesium stearate.

In an embodiment of the invention, by mass of 1 part of the solid dispersion, the pharmaceutical composition may include 1 part of the solid dispersion, 0.65 part of microcrystalline cellulose KG 802, 0.65 part of anhydrous calcium phosphate, 0.078 part of croscarmellose sodium, 0.14 part of hydroxypropyl cellulose, 0.05 part of colloidal silica and 0.03 part of magnesium stearate.

In an embodiment of the invention, by mass of 1 part of the solid dispersion, the pharmaceutical composition may include 1 part of the solid dispersion, 0.66 part of microcrystalline cellulose KG 802, 0.66 part of anhydrous calcium phosphate, 0.078 part of croscarmellose sodium, 0.1 part of hydroxypropyl cellulose, 0.052 part of colloidal silica and 0.04 part of magnesium stearate.

In an embodiment of the invention, by mass of 1 part of the solid dispersion, the pharmaceutical composition may include 1 part of the solid dispersion, 0.65 part of microcrystalline cellulose KG 802, 0.65 part of anhydrous calcium phosphate, 0.084 part of croscarmellose sodium, 0.13 part of hydroxypropyl cellulose, 0.052 part of colloidal silica and 0.03 part of magnesium stearate.

In an embodiment of the invention, by mass of 1 part of the solid dispersion, the pharmaceutical composition may include 1 part of the solid dispersion, 0.61 part of microcrystalline cellulose KG 802, 0.61 part of calcium phosphate, 0.13 part of croscarmellose sodium, 0.16 part of hydroxypropyl cellulose, 0.065 part of colloidal silica and 0.026 part of magnesium stearate.

In an embodiment of the invention, by mass of 1 part of the solid dispersion, the pharmaceutical composition may include 1 part of the solid dispersion, 0.65 part of microcrystalline cellulose KG 802, 0.65 part of anhydrous calcium phosphate, 0.078 part of croscarmellose sodium, 0.13 part of hydroxypropyl cellulose, 0.052 part of colloidal silica and 0.032 part of magnesium stearate.

In an embodiment of the invention, by mass of 1 part of the solid dispersion, the pharmaceutical composition may include 1 part of the solid dispersion, 0.61 part of microcrystalline cellulose KG 802, 0.61 part of anhydrous calcium phosphate, 0.082 part of croscarmellose sodium, 0.13 part of hydroxypropyl cellulose, 0.049 part of colloidal silica and 0.038 part of magnesium stearate.

In an embodiment of the invention, by mass of 1 part of the solid dispersion, the pharmaceutical composition may include 1 part of the solid dispersion, 0.59 part of microcrystalline cellulose KG 802, 0.59 part of anhydrous calcium phosphate, 0.082 part of croscarmellose sodium, 0.13 part of hydroxypropyl cellulose, 0.075 part of colloidal silica and 0.057 part of magnesium stearate.

In an embodiment of the invention, by mass of 1 part of the solid dispersion, the pharmaceutical composition may include 1 part of the solid dispersion, 0.57 parts of microcrystalline cellulose KG 802, 0.57 parts of anhydrous calcium phosphate, 0.082 parts of croscarmellose sodium, 0.13 parts of hydroxypropyl cellulose, 0.14 parts of colloidal silica and 0.057 parts of magnesium stearate.

In an embodiment of the invention, by mass of 1 part of the solid dispersion, the pharmaceutical composition may include 1 part of the solid dispersion, 0.61 part of microcrystalline cellulose KG 802, 0.61 part of anhydrous calcium phosphate, 0.13 part of croscarmellose sodium, 0.16 part of hydroxypropyl cellulose, 0.065 part of colloidal silica and 0.026 part of magnesium stearate.

In an embodiment of the invention, by mass of 1 part of the solid dispersion, the pharmaceutical composition may include 1 part of the solid dispersion, 0.65 part of microcrystalline cellulose KG 802, 0.65 part of anhydrous calcium phosphate, 0.078 part of croscarmellose sodium, 0.14 part of hydroxypropyl cellulose, 0.052 part of colloidal silica and 0.032 part of magnesium stearate.

In an embodiment of the invention, by mass of 1 part of the solid dispersion, the pharmaceutical composition may include 1 part of the solid dispersion, 0.57 parts of microcrystalline cellulose KG 802, 0.57 parts of anhydrous calcium phosphate, 0.082 parts of croscarmellose sodium, 0.13 parts of hydroxypropyl cellulose, 0.14 parts of colloidal silica and 0.057 parts of magnesium stearate.

In an embodiment of the invention, by mass of 1 part of the solid dispersion, the pharmaceutical composition may include 1 part of the solid dispersion, 0.575 parts of microcrystalline cellulose KG 802, 0.575 parts of anhydrous calcium phosphate, 0.13 parts of hydroxypropyl cellulose, 0.08 parts of croscarmellose sodium, 0.13 parts of colloidal silica and 0.042 parts of magnesium stearate.

In an embodiment of the invention, the pharmaceutical composition can be composed of the solid dispersion, the diluent, the dry antisticking agent, the disintegrating agent, the antisticking agent, the glidant and the lubricant.

The invention also provides a pharmaceutical preparation comprising the pharmaceutical composition; The pharmaceutical preparation can be a solid preparation or a powder, granule, tablet, capsule, dropping pill or film.

The invention also provides a preparation method of tablets, which comprises the following steps:
Step 1: the solid dispersion, the diluent and the disintegrating agent are mixed to obtain a mixture, which is sieved and granulated to obtain particles; Optionally mix the dry antisticking agent and the mixture before sieving; Optionally, mix the antisticking agent and glidant with the mixture before sieving; Optionally, the lubricant is added after sieving before granulation;
Step 2: the particles of step 1 are mixed with the lubricant and press the tablet; Optionally, the particles of step 1 are mixed with the antisticking agent and glidant.

The antisticking agent and glidant can be all added in step 1 (the antisticking agent and glidant are no longer added in step 2); Part of the antisticking agent and glidant can also be added in step 1, and the remaining part of the antisticking agent and glidant can also be added in step 2.

The lubricant can be completely added in step 2 (the lubricant is not added in step 1); It is also possible to add part of the lubricant in step 1 and the remaining part of the lubricant in step 2.

In an embodiment of the invention, when part of the antisticking agent and glidant are added in step 1 and the remaining part of the antisticking agent and glidant are added in step 2, the total amount of the antisticking agent and glidant is 100%, and the mass percentage of the antisticking agent and glidant in step 1 is 0.5%˜20% (for example 5.8%).

In an embodiment of the invention, when part of the lubricant is added in step 1 and the remaining part of the lubricant is added in step 2, the total amount of the lubricant is 100%, and the mass percentage of the lubricant in step 1 is 0.1%-10% (for example 1.9%).

In an embodiment of the invention, in step 1, the mixing can be carried out in a mixer; The speed of the mixing can be 15˜21 rpm (for example 18 rpm); The mixing time can be 4 to 10 minutes (for example, 5 minutes).

In an embodiment of the invention, in step 1, the sieve can pass 30˜50 mesh sieve (for example, 40 mesh sieve).

In an embodiment of the invention, in step 1, the granulation can be dry granulation. The pressing wheel pressure of the dry granulation can be 2.0˜10.0 MPa (for example 4.0˜7.0 MPa). The pressing wheel clearance of the dry granulation can be 0.5˜8.0 mm (for example 1.0 mm). The pressing wheel speed of the dry granulation can be 2.0˜10.0 rpm (for example 3.0˜7.0 rpm). The speed of the granulating part of the dry granulation method can be 20˜120 rpm (for example, 30˜90 rpm). The feed shaft speed of the dry granulation can be 10˜120 rpm (for example 18˜90 rpm). The fine screen of dry granulation can be a 0.6˜10.0 mm screen (for example 0.8 mm screen).

In an embodiment of the invention, in step 2, the feed speed of the tablet pressing can be 3 rpm (for example, 5˜30 rpm). The rotating speed of the tablet can be 15˜40 rpm (for example 20˜30 rpm). The tablet thickness scale of the tablet can be 0.1˜7 mm (for example 0.3˜5.0 mm). The feed scale of the tablet can be 7˜18 mm (for example 10.5˜15.5 mm). The main pressure range of the tablet can be 5˜35 KN (for example 10˜25 KN).

The invention also provides a tablet prepared by the preparation method of the tablet.

The invention also provides a coated tablet comprising the pharmaceutical composition.

In an embodiment of the invention, the mass ratio of the pharmaceutical composition and the coating in the coated tablet can be 0.02:1˜0.2:1, or 0.05:1˜0.1:1 (for example 0.08:1).

In an embodiment of the invention, the coating in the coated tablet comprises polyvinyl alcohol, titanium dioxide, talc powder, triethylglycerol and hydroxypropyl methyl cellulose.

In an embodiment of the invention, the coating in the coated tablet is OPADRY®II film coating premix.

The invention also provides a coated tablet, which comprises a tablet prepared by the preparation method of the tablet.

In an embodiment of the invention, the mass ratio of the tablet and the coating in the coated tablet can be 0.02:1˜0.2:1, or 0.05:1˜0.1:1 (for example 0.08:1).

In an embodiment of the invention, the coating in the coated tablet comprises polyvinyl alcohol, titanium dioxide, talc powder, triethylglycerol and hydroxypropyl methyl cellulose.

In an embodiment of the invention, the coating of the coated tablet is OPADRY®II film coating premix.

The invention also provides an application of the solid dispersion as described above in the preparation of drugs for the treatment of related diseases caused by P53 and/or MDM2 abnormalities.

In an embodiment of the invention, the related disease caused by the abnormality of P53 and/or MDM2 is cancer or hyperproliferative disease.

The invention provides a method for treating cancer by administering a therapeutically effective amount of the solid dispersion, the pharmaceutical composition, the tablet or the coated tablet to an individual in need.

In one embodiment of the invention, the cancer is adrenal cortical cancer, advanced cancer, anal cancer, aplastic anemia, cholangiocarcinoma, bladder cancer, bone cancer, bone metastasis, adult brain/CNS tumor, childhood brain/CNS tumor, breast cancer, male breast cancer, childhood cancer, unknown primary cancer, Castleman disease, cervical cancer, Colorectal/rectal cancer, endometrial cancer, esophageal cancer, Ewing family of tumors, eye cancer, gallbladder cancer, gastrointestinal carcinoid tumor, gastrointestinal stromal tumor (GIST), gestational trophoblastic disease, Hodgkin disease, Kaposi sarcoma, renal cancer, laryngeal and hypopharyngeal cancer, adult acute lymphocytic leukemia (ALL) Acute myeloid leukemia (AML), chronic lymphocytic leukemia (CLL), chronic myeloid leukemia (CIVIL), chronic myelomonocytic leukemia (CMML), childhood leukemia, liver cancer, non-small cell lung cancer, small cell lung cancer, lung carcinoid tumor, skin lymphoma, malignant mesothelioma, multiple myeloma, myelodysplastic syndrome, nasal cavity and paranasal sinus cancer Nasopharyngeal carcinoma, neuroblastoma, non-Hodgkin lymphoma, childhood non-Hodgkin's lymphoma, oral and oropharyngeal carcinoma, osteosarcoma, ovarian cancer, pancreatic cancer, penile cancer, pituitary tumor, prostate cancer, retinoblastoma, rhabdomyosarcoma, salivary gland carcinoma, sarcoma—adult soft tissue carcinoma, basal skin cancer and squamous cell carcinoma, skin cancer—melanoma, small bowel cancer, Gastric cancer, testicular cancer, thymic cancer, thyroid cancer, uterine sarcoma, vaginal cancer, vulvar cancer, Waldenstrom macroglobulinemia, or Wilms tumor.

The “optional” in the present invention means that the event or environment described later can but need not occur. The description includes the occasions where the event or environment occurs or does not occur.

The “solid dispersion” of the invention refers to any solid composition with at least two components; It comprises an active ingredient (a compound shown in formula (I)) dispersed in at least one other component (for example, hypromellose acetate succinate).

As shown in formula (I), the pharmaceutically acceptable salt of the compound can be an acid addition salt formed with a pharmaceutically acceptable acid. Examples of acids of pharmaceutically acceptable salts include inorganic acids such as nitric acid, boric acid, hydrochloric acid, hydrobromic acid, sulfuric acid, and phosphoric acid; And organic acids such as oxalic acid, maleic acid, succinic acid, and citric acid. Non limiting examples of salts of compounds of the present invention include, but are not limited to, hydrochloride, hydrobromate, hydroiodate, sulfate, bisulfate, 2-hydroxyethanesulfonate, phosphate, hydrogen phosphate, acetate, adipate, alginate, aspartate, benzoate, bisulfate, butyrate, camphorate, camphorsulfonate, diglucosate Glycerophosphate, hemisulfate, heptanate, caproate, formate, succinate, fumarate, maleate, ascorbate, hydroxyethyl sulfonate, salicylate, methanesulfonate, mesitylene sulfonate, naphthalene sulfonate, nicotinate, 2-naphthalene sulfonate, oxalate, dihydroxynaphthalate, pectinate, persulfate, 3-phenylpropionate Picrate, tervalerate, propionate, trichloroacetate, trifluoroacetate, phosphate, glutamate, bicarbonate, p-toluenesulfonate, undecanoate, lactate, citrate, tartrate, gluconate, methanesulfonate, ethyldisulfonate, benzene sulfonate, and p-toluenesulfonate.

On the basis of not violating the common sense in the art, the above preferred conditions can be arbitrarily combined to obtain the better examples of the invention.

The positive progressive effect of the invention is that the solid dispersion provided by the invention can improve the dissolution of the active ingredient APG-115, the dissolution of some solid dispersions APG-115 of the invention can reach more than 90% (for example, 93.7%, 99.0%, etc.), and has good stability, which can improve the dissolution and dissolution of drugs in gastrointestinal fluid, so as to improve the oral bioavailability. The solid dispersion of the invention shows high plasma exposure in dogs, that is, higher drug peak concentration and higher area under blood concentration curve.

### DESCRIPTION OF DRAWINGS

FIG. 1 shows the curve of oral administration of APG-115ASD and APG-115 hydrate in dogs.

### SPECIFIC EMBODIMENTS

The invention will be further described by way of embodiments, but the invention is not limited to the scope of the examples.

In the following examples, the preparation method of APG-115 hydrate can refer to CN106794171A, and other raw materials are commercially available.

DSC test method: place the test sample in a closed aluminum plate and raise the temperature to 300° C. at the rate of 10° C./min under nitrogen flow to obtain the differential scanning calorimetry (DSC) curve.

Equilibrium solubility test: the suspension sample prepared with water was stirred at 37° C. rpm for 6 hours, then centrifuged to take the supernatant, and the concentration of APG-115 in the supernatant was determined by HPLC.

Drug dissolution test: the dissolution test method II (slurry method) of the Chinese Pharmacopoeia was used. 900 ml of pH 6.8 phosphate solution (0.2% sodium dodecyl sulfate) was used as the dissolution medium, and the rotating speed was 75 revolutions per minute).

In the following examples, the APG-115 hydrate used is APG-115 monohydrate. Other hydrates of APG-115 (such as hemihydrate, monohydrate, dihydrate, trihydrate, tetrahydrate, pentahydrate, hexahydrate, heptahydrate, octahydrate, nonahydrate, decahydrate, undecahydrate, dodecahydrate, etc.) can also be used to prepare APG-115ASD. Of course, ASD can also be prepared by using the salt or crystal form of APG-115.

### EXAMPLE 1

APG-115 hydrate and HPMCAS126G (the mass ratio of APG-115 hydrate and HIPMCAS126G was 1:3, that was, the drug loading was 25%) was weighed. A certain volume of ethyl acetate (which can completely dissolve HPMCAS126G and APG-115 hydrate) was measured, and placed in a glass bottle with thin mouth, HPMCAS126G and APG-115 hydrate was slowly added under magnetic stirring, and stirred until it was completely dissolved; The solution was spray dried (the actual outlet temperature was 4070° C., 5 hours), filtered through a 40 mesh screen to obtain APG-115ASD.

The related substances, equilibrium solubility and drug dissolution of amorphous solid dispersion (APG-115ASD) were tested. The results showed that the related substances were 0.47%; The equilibrium solubility was 0.0898 mg/ml; The drug dissolution rates of 10 min, 30 min and 60 min were 64.5%, 95.2% and 99.0%, respectively.

### EXAMPLE 2

HPMCAS126G in example 1 was replaced by HPMCAS716G, and APG-115ASD was prepared and characterized by the same method as in example 1. The results showed that the related substances were 0.53%; The equilibrium solubility was 0.1099 mg/ml; The drug dissolution rates of 10 min, 30 min and 60 min were 78%, 93.6% and 97.1%, respectively.

### EXAMPLE 3

HPMCAS126G in example 1 was replaced by HPMCAS912G, APG-115ASD was prepared by the same method as in example 1, and DSC scanning, related substances, equilibrium solubility and drug dissolution test were performed. The results showed that DSC scanning showed no obvious endothermic and exothermic peaks; Related substances: 0.50%; The equilibrium solubility was 0.1028 mg/ml; The drug dissolution rates of 10 min, 30 min and 60 min were 80.8%, 97.5% and 101.4%, respectively.

### EXAMPLE 4

HPMCAS126G in example 1 was replaced with HPC Klucel EXF, APG-115ASD was prepared by the same method as in example 1, and DSC scanning and related substances were tested. The results showed that DSC scanning showed no obvious endothermic and exothermic peaks; The related substances were 0.33%.

### EXAMPLE 5

The HPMCAS126G in example 1 was replaced with PVP VA64, and APG-115ASD was prepared and characterized by the same method as in example 1. The results showed that: 0.41% related substances; equilibrium solubility, 0.2384 mg/ml; drug dissolution at 10 min, 30 min, and min was 11.0%, 65.6%, and 91.1%, respectively.

### EXAMPLE 6

The HPMCAS126G in example 1 was replaced with PVP K29/32, and APG-115ASD was prepared and characterized by the same method as in example 1. The results showed that: 0.23% of the related substances; equilibrium solubility, 0.0558 mg/ml; drug dissolution at 10 min, 30 min, and 60 min was 6.0%, 48.4%, and 90.1%, respectively.

### EXAMPLE 7

The HPMCAS126G in example 1 was replaced with PVP S-630, and the APG-115ASD was prepared and characterized by the same procedure as in example 1. The results showed that: 0.85% of the involved substances; equilibrium solubility, 0.0351 mg/ml; drug dissolution at 10 min, 30 min, and 60 min was 13.0%, 76.9%, and 97.7%, respectively.

### EXAMPLE 8

HPMCAS126G in example 1 was replaced with Eudragit L100, and APG-115ASD was prepared and tested for the related substances as well as drug dissolution by the same method as in example 1. The results showed that the drug dissolution was 0.53% for the related substances, and 54.2%, 97.5%, and 98.3% for 10 min, 30 min, and 60 min, respectively.

### EXAMPLE 9

HPMCAS126G in example 1 was replaced with Eudragit S100, and APG-115ASD was prepared and characterized by DSC scanning as well as drug dissolution test using the same method as in example 1. The results showed that DSC scanning showed no obvious endothermic, exothermic peaks, and the drug dissolution was 48.7%, 96.3%, and 99.7% at 10 min, 30 min, and 60 min, respectively.

### EXAMPLE 10

HPMCAS126G in example 1 was replaced by Eudragit L100-55. APG-115 ASD was prepared by the same method as in example 1, and DSC scanning, related substances and drug dissolution tests were performed. The results showed that DSC scanning showed no obvious endothermic, exothermic peaks, and the drug dissolution was 83.7%, 92.8%, and 96.6% at 10 min, 30 min, and 60 min, respectively.

### EXAMPLE 11

APG-115 hydrate and HPMCAS912G (the mass ratio of APG-115 hydrate and HPMCAS912G was 1:4), and colloidal silica (the mass ratio of colloidal silica and APG-115 hydrate is 1:9.8) was weighed. A certain volume of ethyl acetate (it enables complete dissolution of HPMCAS126G, APG-115 hydrate, and colloidal silica) was weighed, placed in a glass bottle with thin mouth, HPMCAS912G and APG-115 hydrate and colloidal silica were slowly added in sequence under magnetic stirring until complete dissolution. The resulting solution was spray dried (actual wind out temperature of 40˜65° C., 5 h); filtered through a 40 mesh screen to obtain APG-115ASD. Amorphous solid dispersion (APG-115ASD) was scanned by DSC and drug dissolution test. The results showed that DSC scan showed no obvious endothermic, exothermic peaks; the drug dissolution was 85.7%, 101.4% and 101.8% at 10 min, 30 min as well as 60 min, respectively.

### EXAMPLE 12

The amount of APG-115 hydrate, HPMCAS912G and colloidal silica in example 11 was changed so that the mass ratio of APG-115 hydrate to HPMCAS912G was 1:3 and the mass ratio of colloidal silica to HPMCAS912G was 1:12.25. APG-115ASD was prepared by the same method as in example 10 and tested for DSC scanning, drug dissolution and total impurities. The results showed that DSC scanning showed no obvious endothermic and exothermic peaks; The drug dissolution rates of 10 min, 30 min and 60 min were 62.3%, 101.1% and 102.2%, respectively; The total impurities at initial, 30 days (60° C.) and 30 days (RH 75%) were 0.67%, 0.96% and 1.38%, respectively.

### EXAMPLE 13

The amount of APG-115 hydrate, HPMCAS912G and colloidal silica in example 11 was changed so that the mass ratio of APG-115 hydrate to HPMCAS912G was 1:2 and the mass ratio of colloidal silica to HPMCAS912G was 1:16.35. APG-115ASD was prepared by the same method as in example 10 and tested for DSC scanning, drug dissolution and total impurities. The results showed that DSC scanning showed no obvious endothermic and exothermic peaks; The drug dissolution rates of 10 min, 30 min and 60 min were 36.3%, 101.4% and 102.3%, respectively; The total impurities at initial, 30 days (60° C.) and 30 days (RH75%) were 0.40%, 1.27% and 0.54%, respectively.

### EXAMPLE 14

The amount of APG-115 hydrate, HPMCAS912G and colloidal silica in example 11 was changed so that the mass ratio of APG-115 hydrate to HPMCAS912G was 1:1, and the mass ratio of colloidal silica to HPMCAS912G was 1:24.5. APG-115ASD was prepared by the same method as example 10, and DSC scanning and drug dissolution test were carried out. The results showed that DSC scanning showed that there were sharp crystal endothermic peaks; The drug dissolution rates of 10 min, 30 min and 60 min were 21.1%, 87.6% and 100.9%, respectively.

### EXAMPLE 15

APG-115 hydrate and hypromellose acetate succinate was weighed. A certain volume of 90% ethanol was measured, placed in a glass bottle with thin mouth, APG-115 hydrate and hypromellose acetate succinate was slowly added under magnetic stirring until it was completely dissolved, the drug containing solution was prepared, the total impurities of the drug containing solution was determined, the drug containing solution was placed for more than 20 hours, and the total impurities of the drug containing solution was determined again. The results showed that the initial total impurities of the drug containing solution were 0.20%, and the total impurities of the solution placed for more than 20 hours were 0.63%.

### EXAMPLE 16

The 90% ethanol in example 14 was replaced with ethanol and tetrahydrofuran (volume ratio: 4:1), the drug containing solution was prepared by the same method as in example 15, and its total impurities were tested. The results showed that the initial total impurity of the drug containing solution was 0.23%, and the total impurity of the solution placed for more than 20 hours was 0.34%.

### EXAMPLE 17

APG-115 hydrate and hypromellose acetate succinate (the mass ratio of APG-115 hydrate and hypromellose acetate succinate was 1:2) was weighed. A certain volume of methyl acetate (which can completely dissolve the hypromellose acetate succinate and APG-115 hydrate) was measured, placed in a glass bottle with thin mouth, APG-115 hydrate and hypromellose acetate succinate was slowly added under magnetic stirring, stirred until it was completely dissolved, a drug containing solution was prepared, the total impurities of the drug containing solution were determine, and the drug containing solution was placed for more than 20 hours, the total impurities of the drug containing solution was determined again. Spray drying was carried out with the drug solution (the actual outlet temperature was 4065° C., 5 hours) and vacuum drying (50 h, 2 hours). The residual solvents of dried APG-115ASD were tested. The results showed that the total impurities in the initial solution were 0.34%, and the total impurities in the solution placed for more than 20 hours were 0.37%; The residual solvent was 0.02%.

### EXAMPLE 18

Methyl acetate in example 17 was replaced with tetrahydrofuran, the drug containing solution was prepared and its total impurities were tested in the same way as in example 17, APG-115ASD was prepared and the solvent residue of dried APG-115ASD was tested. The results showed that the total impurities in the initial solution were 0.25%, and the total impurities in the solution placed for more than 20 hours were 0.27%; The residual solvent was 3.48%.

### EXAMPLE 19

APG-115ASD and microcrystalline cellulose Avicel® PH 102, croscarmellose sodium, colloidal silica and magnesium stearate of example 13 were weighed (in which APG-115ASD, microcrystalline cellulose Avicel® PH 102, croscarmellose sodium, colloidal silica and magnesium stearate accounted for 38.5%, 52.5%, 5%, 3% and 1% of the total materials respectively); APG-115ASD, microcrystalline cellulose Avicel® PH 102, croscarmellose sodium and colloidal silica were shaken and mixed 200 times in clean and dry LDPE Bag after passing through 40 mesh screen for 3 times; 0.5% magnesium stearate passing 60 mesh sieve was added (mass percentage was the mass fraction relative to the total material) to the above mixed powder and shaked it for 60 times; Dry granulation (in which the roller speed was 5 pm, the pressure of the pressure roller was 7-11 MPa, the fine screen was 24 mesh, and the dry particles were collected in clean and dry LDPE Bag); The remaining magnesium stearate passing the 60 mesh screen was added into the dry particles and shaked and mixed in the LDPE bag for 60 times to obtain the total mixed powder; The total mixed powder was stamped on a single stamping machine with a shallow arc circle with a diameter of 10 mm to make a plain tablet with a weight of 400 mg. The parameters and drug dissolution of Plain tablet were tested, and the results were shown in Table 1.

TABLE 1
Parameters of plain tablets

| Surface | | | | |
|---|---|---|---|---|
| condition of | Average | Average | Disintegration | Friability |
| the plain tablet | hardness | thickness | time | (200 rpm) |
| White with | 103.1N | 4.82 mm | 1 min 03 | 0.31% |
| occasional spots | | | seconds | |

Drug dissolution rate (%)

| | | | | | | |
|---|---|---|---|---|---|---|
| 5 min | 10 min | 20 min | 30 min | 45 min | 60 min | 90 min |
| 66 | 86 | 94 | 95 | 94 | 95 | 94 |

### EXAMPLE 20

APG-115ASD and microcrystalline cellulose Avicel® PH 102, pregelatinized starch, croscarmellose sodium, colloidal silica and magnesium stearate of example 13 were weighed (in which APG-115ASD and microcrystalline cellulose Avicel® PH 102, pregelatinized starch, croscarmellose sodium, colloidal silica and magnesium stearate accounted for 38.5%, 35%, 17.5%, 5%, 3% and 1% of the total materials respectively); APG-115ASD, microcrystalline cellulose Avicel® PH 102, pregelatinized starch, croscarmellose sodium and colloidal silica were placed in a clean and dry LDPE bag and shaked for 200 times after passing through a 40 mesh screen for 3 times; 0.5% magnesium stearate passing 60 mesh sieve (mass percentage is the mass fraction relative to the total material) was added to the above mixed powder and shaked it for 60 times; Dry granulation (in which the roller speed was 5 rpm, the pressure of the pressing roller was 7-11 MPa, the fine screen was 24 mesh, and the dry particles were collected in clean and dry LDPE Bag); The remaining magnesium stearate passing the 60 mesh screen was added into the dry particles and shaked and mixed in the LDPE bag for 60 times to obtain the total mixed powder; The total mixed powder was stamped on a single stamping machine with a shallow arc circle with a diameter of 10 mm to make a plain tablet with a weight of 400 mg. The parameters and drug dissolution of Plain tablet were tested, and the results were shown in Table 2.

TABLE 2
Parameters of plain tablets

| Surface | | | | |
|---|---|---|---|---|
| condition of | Average | Average | Disintegration | Friability |
| the plain tablet | hardness | thickness | time | (200 rpm) |
| White with | 66.1N | 4.90 mm | 42 seconds | 0.50% |
| occasional spots | | | | |

Drug dissolution rate (%)

| | | | | | | |
|---|---|---|---|---|---|---|
| 5 min | 10 min | 20 min | 30 min | 45 min | 60 min | 90 min |
| 76 | 94 | 100 | 101 | 99 | 100 | 100 |

### EXAMPLE 21

APG-115ASD and microcrystalline cellulose Avicel® PH 102, calcium phosphate, croscarmellose sodium, colloidal silica and magnesium stearate of example 13 were weighed (in which APG-115ASD, microcrystalline cellulose Avicel® PH 102, calcium phosphate, croscarmellose sodium, colloidal silica and magnesium stearate accounted for 38.5%, 26.25%, 26.25%, 5%, 3% and 1% of the total materials respectively); APG-115ASD, microcrystalline cellulose Avicel® PH 102, calcium phosphate, croscarmellose sodium and colloidal silica were placed in a clean and dry LDPE bag and shaked for 200 times after passing through a 40 mesh screen for 3 times; 0.5% magnesium stearate passing 60 mesh sieve (mass percentage is the mass fraction relative to the total material) was added to the above mixed powder and shaked for 60 times; Dry granulation (in which the roller speed was 5 rpm, the pressure of the pressing roller is 7-11 MPa, the fine screen was 24 mesh, and the dry particles were collected in clean and dry LDPE Bag); The remaining magnesium stearate passing the 60 mesh screen was added into the dry particles and shaked and mixed in the LDPE bag for 60 times to obtain the total mixed powder; The total mixed powder was stamped on a single stamping machine with a shallow arc circle with a diameter of 10 mm to make a plain tablet with a weight of 400 mg. The parameters and drug dissolution of Plain tablet were tested, and the results were shown in Table 3.

TABLE 3
Parameters of plain tablets

| Surface condition | Average | Average | Disintegration | Friability |
|---|---|---|---|---|
| of the plain tablet | hardness | thickness | time | (200 rpm) |
| White with | 129.7 N | 4.60 mm | 35 seconds | 0.12% |
| occasional spots | | | | |

Drug dissolution rate (%)

| 5 min | 10 min | 20 min | 30 min | 45 min | 60 min | 90 min |
|---|---|---|---|---|---|---|
| 68 | 84 | 91 | 94 | 95 | 97 | 97 |

### EXAMPLE 22

APG-115ASD, microcrystalline cellulose KG 802, calcium phosphate, croscarmellose sodium, hydroxypropyl cellulose, colloidal silica and magnesium stearate of example 13 were weighed (in which APG-115ASD, microcrystalline cellulose KG 802, calcium phosphate, croscarmellose sodium, hydroxypropyl cellulose, colloidal silica and magnesium stearate accounted for 38.5%, 23.5%, 23.5%, 5%, 6%, 2.5% and 1% of the total materials respectively); APG-115ASD, microcrystalline cellulose KG 802, calcium phosphate, croscarmellose sodium, hydroxypropyl cellulose and colloidal silica were passed through 40 mesh screen for 3 times and then placed in a clean and dry LDPE bag for shaking and mixing for 200 times; 0.5% magnesium stearate passing through 60 mesh screen (mass percentage relative to the total material) was added to the above mixed powder and shaked and mixed for 60 times; Dry granulation (the rotating speed of roller was 5 rpm, the pressure of roller was 7-11 mpa, the fine screen mesh was 24, and the dry particles were collected in clean and dry LDPE Bag), and the amount of fine powder (<80 mesh) was more than 20%; The remaining magnesium stearate passing the 60 mesh screen was added into the above dry particles and shaked and mixed in the LDPE bag for 60 times to obtain the total mixed powder; The total mixed powder was pressed into 400 mg plain tablets with a circular die with a diameter of 9.5 mm on a single stamping machine, and then the OPADRY II 85f620079 (the mass of OPADRY II 85f620079 was 3% of that of plain tablets) film coating premix was used to coat the plain tablets. The parameters of the plain tablets and the drug dissolution of the coated tablets were tested. The results are shown in Table 4.

TABLE 4
Parameters of plain tablets

| Surface condition | Average | Disintegration | Friability |
|---|---|---|---|
| of the plain tablet | hardness | time | (200 rpm) |
| White with | 137 N | 55 seconds | 0.2% |
| occasional spots | | | |

Drug dissolution rate (%)

| 5 min | 10 min | 20 min | 30 min | 45 min | 60 min | 90 min |
|---|---|---|---|---|---|---|
| 54 | 77 | 90 | 93 | 93 | 93 | 99 |

### EXAMPLE 23

APG-115ASD, microcrystalline cellulose KG 802, anhydrous calcium hydrogen phosphate, croscarmellose sodium, hydroxypropyl cellulose, colloidal silica and magnesium stearate of example 13 were weighed (the mass proportions of APG-115ASD, microcrystalline cellulose KG 802, anhydrous calcium hydrogen phosphate, croscarmellose sodium, hydroxypropyl cellulose, colloidal silica and magnesium stearate in the total materials were 38.5%, 25%, 25%, 3%, 5.25%, 2% and 1.25% respectively); APG-115ASD, microcrystalline cellulose KG 802, anhydrous calcium hydrogen phosphate, croscarmellose sodium, hydroxypropyl cellulose and colloidal silica were passed through a 40 mesh screen for 3 times, and then placed in a clean and dry LDPE bag for shaking and mixing for 200 times; 0.75% of magnesium stearate passing a 60 mesh screen (the mass percentage was the mass fraction relative to the total material) was added into the above mixed powder and shaked for 60 times; dry granulation (in which the roller speed was 5 rpm, the pressing wheel pressure was 7-11 MPa, the fine screen mesh was 24, and the dry particles were collected in clean and dry LDPE Bag), and the amount of fine powder (<80 mesh) 18%; The remaining magnesium stearate passing the 60 mesh screen was added into the above dry particles and shaked and mixed in the LDPE bag for 60 times to obtain the total mixed powder; The total mixed powder was pressed into 400 mg plain tablets with a circular die with a diameter of 9.5 mm on a single stamping machine, and then OPADRY II 85f620079 (the mass of OPADRY II 85f620079 was 3% of the plain tablets) film coating premix was used to coat the plain tablets to obtain the coated tablets. The parameters of the plain tablets and the drug dissolution of the coated tablets were tested. The results were shown in Table 5.

TABLE 5
Parameters of plain tablets

| Surface condition | Average | Disintegration | Friability |
|---|---|---|---|
| of the plain tablet | hardness | time | (200 rpm) |
| White with | 135 N | 40 seconds | 0.2% |
| occasional spots | | | |

Drug dissolution rate (%)

| 5 min | 10 min | 20 min | 30 min | 45 min | 60 min | 90 min |
|---|---|---|---|---|---|---|
| 47 | 72 | 91 | 95 | 96 | 96 | 101 |

### EXAMPLE 24

APG-115ASD, microcrystalline cellulose KG 802, anhydrous calcium hydrogen phosphate, croscarmellose sodium, hydroxypropyl cellulose, colloidal silica and magnesium stearate of example 13 were weighed (the mass proportions of APG-115ASD, microcrystalline cellulose KG 802, anhydrous calcium hydrogen phosphate, hydroxypropyl cellulose, croscarmellose sodium, colloidal silica and magnesium stearate in the total materials were 38.5%, 25.5%, 25.5%, 4%, 3%, 2% and 1.5% respectively); APG-115ASD, microcrystalline cellulose KG 802, anhydrous calcium hydrogen phosphate, croscarmellose sodium, hydroxypropyl cellulose and colloidal silica were passed through 40 mesh screen for 3 times, and then placed in clean and dry LDPE bag for shaking and mixing for 200 times; 1% magnesium stearate passing 60 mesh screen (mass percentage was relative to the mass fraction of the total material) was added into the above mixed powder and shaked for 60 times; Dry granulation (in which the roller speed was 5 rpm, the pressing wheel pressure was 7-11 MPa, the fine screen was 24 mesh, and the dry particles were collected in clean and dry LDPE Bag); The remaining magnesium stearate passing the 60 mesh screen was added into the above dry particles and shaked and mixed in the LDPE bag for 60 times to obtain the total mixed powder; The total mixed powder was pressed into 400 mg plain tablets with a circular die with a diameter of 9.5 mm on a single stamping machine, and then OPADRY II 85f80010 (the mass of OPADRY II 85f80010 is 3% of that of plain tablets) film coating premix was used to coat the plain tablets to obtain the coated tablets. The parameters of the plain tablets were tested, and the results were shown in Table 6.

TABLE 6
Parameters of plain tablets

| Average hardness | Plain | Dis- | |
|---|---|---|---|
| (compression | tablet | integration | Friability |
| pressure 10 kN) | thickness | time | (200 rpm) |
| 120 N | 5.21 mm | 2 min 29 | 0.46% |
| | | seconds | |

### EXAMPLE 25

APG-115ASD, microcrystalline cellulose KG 802, anhydrous calcium hydrogen phosphate, croscarmellose sodium, colloidal silica and magnesium stearate of example 13 were weighed (wherein the mass proportions of APG-115ASD, microcrystalline cellulose KG 802, anhydrous calcium hydrogen phosphate, croscarmellose sodium, colloidal silica and magnesium stearate in the total material were 38.5%, 27.5%, 27.5%, 3%, 2% and 1.5%, respectively); APG-115ASD, microcrystalline cellulose KG 802, anhydrous calcium hydrogen phosphate, croscarmellose sodium and colloidal silica were passed through a 40 mesh screen for 3 times, and then placed in a clean and dry LDPE bag for shaking and mixing for 200 times; 1% magnesium stearate passing 60 mesh sieve (mass percentage is the mass fraction relative to the total material) was added to the above mixed powder and shake it for 60 times; Dry granulation (in which the roller speed was 5 rpm, the pressure of the pressing roller is 7-11 MPa, the fine screen was 24 mesh, and the dry particles were collected in clean and dry LDPE Bag); The remaining magnesium stearate passing the 60 mesh screen was added into the dry particles and shaked and mixed in the LDPE bag for 60 times to obtain the total mixed powder; The total mixed powder was pressed into 400 mg plain tablets with a circular die with a diameter of 9.5 mm on a single tablet press, and then the plain tablets were coated with OPADRY II 85f80010 (the mass of OPADRY II 85f80010 was 3% of the plain tablets) film coating premix to obtain the coated tablets. The parameters of plain tablets were tested, and the results were shown in Table 7.

TABLE 7
Parameters of plain tablets

| Average hardness | Plain | Dis- | |
|---|---|---|---|
| (compression | tablet | integration | Friability |
| pressure 10 kN) | thickness | time | (200 rpm) |
| 92.4 N | 5.28 mm | 45 seconds | 0.34% |

### EXAMPLE 26

APG-115ASD, microcrystalline cellulose KG 802, anhydrous calcium hydrogen phosphate, croscarmellose sodium, hydroxypropyl cellulose, colloidal silica and magnesium stearate of example 13 were weighed (the mass proportions of APG-115ASD, microcrystalline cellulose KG 802, anhydrous calcium hydrogen phosphate, hydroxypropyl cellulose, croscarmellose sodium, colloidal silica and magnesium stearate in the total materials were 38.5%, 25%, 25%, 5%, 3.25%, 2% and 1.25% respectively); APG-115ASD, microcrystalline cellulose KG 802, anhydrous calcium hydrogen phosphate, croscarmellose sodium, hydroxypropyl cellulose and colloidal silica were passed through a 40 mesh screen for 3 times, and then placed in a clean and dry LDPE bag for shaking and mixing for 200 times; 0.75% of magnesium stearate passing a 60 mesh screen (the mass percentage is the mass fraction relative to the total material) was added into the above mixed powder and shaked for 60 times; Dry granulation (in which the roller speed was 5 rpm, the pressing wheel pressure was 7-11 MPa, the fine screen was 24 mesh, and the dry particles were collected in clean and dry LDPE Bag); The remaining magnesium stearate passing the 60 mesh screen was added into the above dry particles and shaked and mixed in the LDPE bag for 60 times to obtain the total mixed powder; The total mixed powder was pressed into 400 mg plain tablets with a circular die with a diameter of 9.5 mm on a single tablet press. The parameters of plain tablets and drug dissolution were tested, and the results were shown in Table 8.

TABLE 8
Parameters of plain tablets

| Average hardness | | |
|---|---|---|
| (compression | Disintegration | Friability |
| pressure 10 kN) | time | (200 rpm) |
| 136.9 N | 1 min 29 seconds | 0.09% |

Drug dissolution rate (%)

| 5 min | 10 min | 20 min | 30 min | 45 min | 60 min | 90 min |
|---|---|---|---|---|---|---|
| 41 | 65 | 84 | 90 | 91 | 91 | 92 |

### EXAMPLE 27

APG-115ASD, silicified microcrystalline cellulose, crospovidone and magnesium stearate of example 13 were weighed (wherein the mass proportions of APG-115ASD, silicified microcrystalline cellulose, crospovidone and magnesium stearate in the total material were 51.75%, 39.5%, 8% and 0.75% respectively); After APG-115ASD, silicified microcrystalline cellulose and crospovidone was passing through 40 mesh screen for 3 times and placed in a clean and dry LDPE bag for shaking and mixing for 200 times; Magnesium stearate passing a 60 mesh sieve was added into the mixed powder and shaked for 60 times; The total mixed powder was pressed into 400 mg plain tablets by a circular die with a diameter of 9.5 mm on a single tablet press. The disintegration time and drug dissolution of plain tablet were tested. The results showed that the disintegration time was 7 minutes, and the drug dissolution were shown in Table 9.

TABLE 9
Drug dissolution (%)

| 5 min | 10 min | 20 min | 30 min | 45 min | 60 min | 90 min |
|---|---|---|---|---|---|---|
| 19 | 41 | 71 | 85 | 93 | 95 | 96 |

### EXAMPLE 28

APG-115ASD, microcrystalline cellulose KG 802, calcium phosphate, croscarmellose sodium, hydroxypropyl cellulose, colloidal silica and magnesium stearate of example 13 were weighed (APG-115ASD, microcrystalline cellulose KG 802, calcium phosphate, croscarmellose sodium, hydroxypropyl cellulose, colloidal silica and magnesium stearate account for 38.5%, 23.5%, 23.5%, 5%, 6%, 2.5% and 1% of the total materials respectively); APG-115ASD, microcrystalline cellulose KG 802, calcium phosphate, croscarmellose sodium, hydroxypropyl cellulose and colloidal silica were passed through a 40 mesh screen for three times and placed in a clean and dry LDPE bag for shaking and mixing for 200 times; 0.5% magnesium stearate passing through a 60 mesh screen (mass percentage is the mass fraction of the total material) was added to the above mixed powder and shaked and mixed for 60 times; Dry granulation (the rotating speed of the roller was 5 rpm, the pressure of the pressure roller was 7-11 mpa, the fine screen was 24 mesh, and the dry particles were collected in clean and dry LDPE Bag); The remaining magnesium stearate passing the 60 mesh screen was added into the above dry particles and shaked and mixed in the LDPE bag for 60 times to obtain the total mixed powder; The total mixed powder was pressed into 400 mg plain tablets with a circular die with a diameter of 9.5 mm on a single tablet press. The parameters of plain tablets and drug dissolution were tested, and the results were shown in Table 10.

TABLE 10
Parameters of plain tablets

| Average hardness | Disintegration time | Friability (200 rpm) |
|---|---|---|
| 137 N | 55 seconds | 0.2% |

Drug dissolution rate (%)

| 5 min | 10 min | 20 min | 30 min | 45 min | 60 min | 90 min |
|---|---|---|---|---|---|---|
| 54 | 77 | 90 | 93 | 93 | 93 | 99 |

### EXAMPLE 29

APG-115ASD, microcrystalline cellulose KG 802, anhydrous calcium hydrogen phosphate, croscarmellose sodium, hydroxypropyl cellulose, colloidal silica and magnesium stearate of example 13 were weighed (the mass proportions of APG-115ASD, microcrystalline cellulose KG 802, anhydrous calcium hydrogen phosphate, croscarmellose sodium, hydroxypropyl cellulose, colloidal silica and magnesium stearate in the total materials were 38.5%, 25%, 25%, 3.25%, 5%, 2% and 1.25% respectively); APG-115ASD, microcrystalline cellulose KG 802, anhydrous calcium hydrogen phosphate, croscarmellose sodium, hydroxypropyl cellulose and colloidal silica were passed through 40 mesh screen for 3 times, and then placed in clean and dry LDPE bag for shaking and mixing for 200 times; 0.75% magnesium stearate passing 60 mesh screen (mass percentage is relative to the mass fraction of the total material) was added into the above mixed powder and shaked for 60 times; Dry granulation (in which the roller speed was 5 rpm, the pressing wheel pressure was 7-11 MPa, the fine screen was 24 mesh, and the dry particles were collected in clean and dry LDPE Bag); The remaining magnesium stearate passing the 60 mesh screen was added into the above dry particles and shaked and mixed in the LDPE bag for 60 times to obtain the total mixed powder; The total mixed powder was pressed into 400 mg plain tablets with a circular die with a diameter of 9.5 mm on a single tablet press. The parameters of plain tablets and drug dissolution were tested, and the results were shown in Table 11.

TABLE 11
Parameters of plain tablets

| Average hardness | Disintegration time | Friability (200 rpm) |
|---|---|---|
| 136.9 N | 1 min 29 seconds | 0.09% |

Drug dissolution rate (%)

| 5 min | 10 min | 20 min | 30 min | 45 min | 60 min | 90 min |
|---|---|---|---|---|---|---|
| 41 | 65 | 84 | 90 | 91 | 91 | 92 |

### EXAMPLE 30

APG-115ASD, microcrystalline cellulose KG 802, anhydrous calcium hydrogen phosphate, croscarmellose sodium, hydroxypropyl cellulose, colloidal silica and magnesium stearate of example 13 were weighed (the mass proportions of APG-115ASD, microcrystalline cellulose KG 802, anhydrous calcium hydrogen phosphate, croscarmellose sodium, hydroxypropyl cellulose, colloidal silica and magnesium stearate in the total materials were 39.65%, 24.31%, 24.31%, 3.26%, 5.01%, 1.96% and 1.50% respectively); APG-115ASD, microcrystalline cellulose KG 802, anhydrous calcium hydrogen phosphate, hydroxypropyl cellulose, croscarmellose sodium and colloidal silica were placed in a mixer for mixing at a mixing speed of 18 rpm for 5 minutes; the obtained material was sieved through 40 mesh sieve, and then 0.75% magnesium stearate (mass percentage was the mass fraction relative to the total material) was added to mix, the mixing speed was 18 rpm, the mixing time was 5 minutes, and the obtained materials was placed in the dry granulator for dry granulation (the pressure of the pressure roller was 4.0-7.0 MPa, the clearance of the pressure roller was 1.0-5.0 mm, the speed of the pressure roller was 3.0-7.0 rpm, the speed of the granulating parts was 30-90 rpm, the speed of the feed shaft was 18-90 rpm, and the fine screen was 0.8 mm) to collect the obtained particles; The remaining magnesium stearate was added to the obtained particles and mixed for lubrication; The tablets were pressed with a circular die with a diameter of 10 mm (the feed speed was 5-30 rpm, the tablet pressing speed was 20-30 rpm, the tablet thickness scale was 0.3-5.0 mm, the feed scale was 10.5-15.5 mm, and the main pressure range was 10-25 KN) to get plain tablets. The parameters of plain tablets and drug dissolution were tested, and the results were shown in Table 12.

TABLE 12
Parameters of plain tablets

| Average hardness | Disintegration time | Friability (200 rpm) |
|---|---|---|
| 143 N | 43 seconds | 0.14% |

Drug dissolution rate (%)

| 5 min | 10 min | 20 min | 30 min | 45 min | 60 min | 90 min |
|---|---|---|---|---|---|---|
| 54 | 79 | 94 | 96 | 97 | 98 | 100 |

### EXAMPLE 31

APG-115ASD, microcrystalline cellulose KG 802, anhydrous calcium hydrogen phosphate, croscarmellose sodium, hydroxypropyl cellulose, colloidal silica and magnesium stearate of example 13 were weighed (the mass proportions of APG-115ASD, microcrystalline cellulose KG 802, anhydrous calcium hydrogen phosphate, croscarmellose sodium, hydroxypropyl cellulose, colloidal silica and magnesium stearate in the total materials were 39.55%, 23.5%, 23.5%, 3.25%, 5%, 2.95% and 2.25% respectively); APG-115ASD, microcrystalline cellulose KG 802, anhydrous calcium hydrogen phosphate, hydroxypropyl cellulose, croscarmellose sodium and 1.45% colloidal silica (mass percentage relative to the total material) were placed in the mixer for mixing at a mixing speed of 18 rpm for 5 minutes; the obtained material was sieved through 40 mesh sieve, and then 0.75% magnesium stearate was added (mass percentage was the mass fraction relative to the total material) mix, the mixing speed was 18 rpm, the mixing time was 5 minutes, and the obtained material was placed in the dry granulator for dry granulation (the pressure of the pressure roller was 4.0-7.0 MPa, the clearance of the pressure roller was 1.0-5.0 mm, the speed of the pressure roller was 3.0-7.0 rpm, the speed of the granulating parts was 30-90 rpm, the speed of the feed shaft was 18-90 rpm, and the fine screen was 0.8 mm) to collect the obtained particles; the remaining colloidal silica and the remaining magnesium stearate was added to the obtained particles for mixed lubrication; the tablets were pressed with a circular die with a diameter of 10 mm (the feed speed was 5-30 rpm, the tablet pressing speed was 20-30 rpm, the tablet thickness scale was 0.3-5.0 mm, the feed scale was 10.5-15.5 mm, and the main pressure range was 10-25 KN) to get plain tablets. The parameters of plain tablets and drug dissolution were tested, and the results were shown in Table 13.

TABLE 13
Parameters of plain tablets

| Average hardness | Disintegration time | Friability (200 rpm) |
|---|---|---|
| 101 N | 31 seconds | 0.35% |

Drug dissolution rate (%)

| 5 min | 10 min | 20 min | 30 min | 45 min | 60 min | 90 min |
|---|---|---|---|---|---|---|
| 46 | 71 | 90 | 94 | 95 | 96 | 100 |

### EXAMPLE 32

APG-115ASD, microcrystalline cellulose KG 802, anhydrous calcium hydrogen phosphate, croscarmellose sodium, hydroxypropyl cellulose, colloidal silica and magnesium stearate of example 13 were weighed (the mass proportions of APG-115ASD, microcrystalline cellulose KG 802, anhydrous calcium hydrogen phosphate, croscarmellose sodium, hydroxypropyl cellulose, colloidal silica and magnesium stearate in the total materials were 39.35%, 22.39%, 22.39%, 3.23%, 4.98%, 5.42% and 2.24% respectively); APG-115ASD, microcrystalline cellulose KG 802, anhydrous calcium hydrogen phosphate, hydroxypropyl cellulose, croscarmellose sodium and 1.19% colloidal silica (mass percentage relative to the total material) were placed in the mixer for mixing at a mixing speed of 18 rpm for 5 minutes; the obtained material was sieved through 40 mesh sieve, and then 0.75% magnesium stearate was added (mass percentage was the mass fraction relative to the total material) to mix, the mixing speed was 18 rpm, the mixing time was 5 minutes, and the obtained material was placed in the dry granulator for dry granulation (the pressure of the pressure roller was 4.0-7.0 MPa, the clearance of the pressure roller was 1.0-5.0 mm, the speed of the pressure roller was 3.0-7.0 rpm, the speed of the granulating parts was 30-90 rpm, the speed of the feed shaft was 18-90 rpm, and the fine screen was 0.8 mm) to collect the obtained particles; the remaining colloidal silica and the remaining magnesium stearate was added to the obtained particles for mixed lubrication; the tablets were pressed with a circular die with a diameter of 10 mm (the feed speed was 5-30 rpm, the tablet pressing speed was 20-30 rpm, the tablet thickness scale was 0.3-5.0 mm, the feed scale was 10.5-15.5 mm, and the main pressure range was 10-25 KN) to get plain tablets. The parameters of plain tablets and drug dissolution were tested, and the results were shown in Table 14.

TABLE 14
Parameters of plain tablets

| Average hardness | Disintegration time | Friability (200 rpm) |
|---|---|---|
| 160 N | 60 seconds | 0.37% |

Drug dissolution rate (%)

| 5 min | 10 min | 20 min | 30 min | 45 min | 60 min | 90 min |
|---|---|---|---|---|---|---|
| 35 | 60 | 84 | 93 | 96 | 97 | 101 |

### EXAMPLE 33

APG-115ASD, microcrystalline cellulose KG 802, anhydrous calcium hydrogen phosphate, croscarmellose sodium, hydroxypropyl cellulose, colloidal silica and magnesium stearate of example 13 were weighed (APG-115ASD, microcrystalline cellulose KG 802, anhydrous calcium hydrogen phosphate, croscarmellose sodium, hydroxypropyl cellulose, colloidal silica and magnesium stearate account for 38.5%, 23.5%, 23.5%, 5%, 6%, 2.5% and 1% of the total materials respectively); APG-115ASD, microcrystalline cellulose KG 802, anhydrous calcium hydrogen phosphate, hydroxypropyl cellulose, croscarmellose sodium and colloidal silica were placed in a mixer for mixing at a mixing speed of 18 rpm for 5 minutes; the obtained material was sieved through 40 mesh sieve, and then 0.5% magnesium stearate (mass percentage was the mass fraction relative to the total material) was added to mix, the mixing speed was 18 rpm, the mixing time was 5 minutes, and the obtained materials was placed in the dry granulator for dry granulation (the pressure of the pressure roller was 4.0-7.0 MPa, the clearance of the pressure roller was 1.0-5.0 mm, the speed of the pressure roller was 3.0-7.0 rpm, the speed of the granulating parts was 30-90 rpm, the speed of the feed shaft was 18-90 rpm, and the fine screen was 0.8 mm), the fluidity was poor, the screen was adheres, and the obtained particles were collected; the remaining magnesium stearate was added to the obtained particles and mixed for lubrication; the tablets were pressed with a circular die with a diameter of 10 mm (the feed speed was 5-30 rpm, the tablet pressing speed was 20-30 rpm, the tablet thickness scale was 0.3-5.0 mm, the feed scale was 10.5-15.5 mm, and the main pressure range was 10-25 KN) to get plain tablets. The parameters of plain tablets and drug dissolution were tested, and the results were shown in Table 15.

TABLE 15
Parameters of plain tablets

| Average | Surface condition | Disintegration | Friability |
|---|---|---|---|
| hardness | of the plain tablet | time | (200 rpm) |
| 137 N | White with | 55 seconds | 0.2% |
| | occasional spots | | |

Drug dissolution rate (%)

| 5 min | 10 min | 20 min | 30 min | 45 min | 60 min | 90 min |
|---|---|---|---|---|---|---|
| 54 | 77 | 90 | 93 | 93 | 93 | 99 |

### EXAMPLE 34

APG-115ASD, microcrystalline cellulose KG 802, anhydrous calcium hydrogen phosphate, croscarmellose sodium, hydroxypropyl cellulose, colloidal silica and magnesium stearate of example 13 were weighed (the mass proportions of APG-115ASD, microcrystalline cellulose KG 802, anhydrous calcium hydrogen phosphate, croscarmellose sodium, hydroxypropyl cellulose, colloidal silica and magnesium stearate in the total materials were 38.5%, 25%, 25%, 3%, 5.25%, 2% and 1.25% respectively); APG-115ASD, microcrystalline cellulose KG 802, anhydrous calcium hydrogen phosphate, hydroxypropyl cellulose, croscarmellose sodium and colloidal silica were placed in a mixer for mixing at a mixing speed of 18 rpm for 5 minutes; the obtained material was sieved through 40 mesh sieve, and then 0.75% magnesium stearate (mass percentage was the mass fraction relative to the total material) was added to mix, the mixing speed was 18 rpm, the mixing time was 5 minutes, and the obtained materials was placed in the dry granulator for dry granulation (the pressure of the pressure roller was 4.0-7.0 MPa, the clearance of the pressure roller was 1.0-5.0 mm, the speed of the pressure roller was 3.0-7.0 rpm, the speed of the granulating parts was 30-90 rpm, the speed of the feed shaft was 18-90 rpm, and the fine screen was 0.8 mm) to collected the obtained particles; the remaining magnesium stearate was added to the obtained particles and mixed for lubrication; the tablets were pressed with a circular die with a diameter of 10 mm (the feed speed was 5-30 rpm, the tablet pressing speed was 20-30 rpm, the tablet thickness scale was 0.3-5.0 mm, the feed scale was 10.5-15.5 mm, and the main pressure range was 10-25 KN) to get plain tablets. The parameters of plain tablets and drug dissolution were tested, and the results were shown in Table 16.

TABLE 16
Parameters of plain tablets

| Average | Surface condition | Disintegration | Friability |
|---|---|---|---|
| hardness | of the plain tablet | time | (200 rpm) |
| 135 N | White with | 40 seconds | 0.2% |
| | occasional spots | | |

Drug dissolution rate (%)

| 5 min | 10 min | 20 min | 30 min | 45 min | 60 min | 90 min |
|---|---|---|---|---|---|---|
| 47 | 72 | 91 | 95 | 96 | 96 | 101 |

### EXAMPLE 35

APG-115ASD, microcrystalline cellulose KG 802, anhydrous calcium hydrogen phosphate, croscarmellose sodium, hydroxypropyl cellulose, colloidal silica and magnesium stearate of example 13 were weighed (the mass proportions of APG-115ASD, microcrystalline cellulose KG 802, anhydrous calcium hydrogen phosphate, croscarmellose sodium, hydroxypropyl cellulose, colloidal silica and magnesium stearate in the total materials were 39.35%, 22.39%, 22.39%, 3.23%, 4.98%, 5.42% and 2.24% respectively); APG-115ASD, microcrystalline cellulose KG 802, anhydrous calcium hydrogen phosphate, hydroxypropyl cellulose, croscarmellose sodium and 1.19% colloidal silica (mass percentage relative to the total material) were placed in the mixer for mixing, the mixing speed was 18 rpm, and mixing time was 5 minutes; the obtained material was passed through 40 mesh sieve, the remaining colloidal silica and 0.75% magnesium stearate (mass percentage is the mass fraction relative to the total material) was added for mixing, the mixing speed was 18 rpm, and mixing time was 5 minutes, and the obtained material was placed in the dry granulator for dry granulation (the pressure of the pressure roller was 4.0-7.0 MPa, the clearance of the pressure roller was 1.0-5.0 mm, the speed of the pressure roller was 3.0-7.0 rpm, the speed of the granulating part was 30-90 rpm, the speed of the feed shaft was 18-90 rpm, and the fine screen was 0.8 mm), the obtained particles were collected; the remaining magnesium stearate was added to the obtained particles and mixed for lubrication; the tablets was pressed with a circular die with a diameter of 10 mm (the feed speed was 5-30 rpm, the tablet pressing speed was 20-30 rpm, the tablet thickness scale was 0.3-5.0 mm, the feed scale was 10.5-15.5 mm, and the main pressure range was 10-25 KN); the plain tablets were obtained. The parameters and drug dissolution of the plain tablets were tested, and the results were shown in Table 17.

TABLE 17
Parameters of plain tablets

| Average | Surface condition | Disintegration | Friability |
|---|---|---|---|
| hardness | of the plain tablet | time | (200 rpm) |
| 160 N | White with | 60 seconds | 0.37% |
| | occasional spots | | |

Drug dissolution rate (%)

| 5 min | 10 min | 20 min | 30 min | 45 min | 60 min | 90 min |
|---|---|---|---|---|---|---|
| 35 | 60 | 84 | 93 | 96 | 97 | 101 |

### EXAMPLE 36

OPADRY II 89k680001-CN plain tablets were used for film coating, in which the coating weight gain was 3%. The drug dissolution of the coated tablets was tested, and the test results were shown in Table 18.

TABLE 18
Drug dissolution rate (%)

| 5 min | 10 min | 20 min | 30 min | 45 min | 60 min | 90 min |
|---|---|---|---|---|---|---|
| 35 | 60 | 84 | 93 | 96 | 97 | 101 |

### EXAMPLE 37

725.2 g of hypromellose acetate succinate (HPMCAS912G) and 359.8 g of APG-115 hydrate was dissolved in 18.4571 of methyl acetate, then 22.4 g of colloidal silica was added and stirred evenly. The spray dryer was set up, and the equipment was stable until the spray was dried for 5 hours. The methyl acetate was collected and the spray dried powder was collected. The spray dried powder was dried for two times (vacuum desiccation and drying for 2 hours) until the residual amount of powder solvent did not exceed 5000 ppm to obtain APG-115ASD powder. 630 g of microcrystalline cellulose KG 802, 630 g of anhydrous calcium phosphate, 140 g of hydroxypropyl cellulose, 91 g of croscarmellose sodium, 33.6 g of colloidal silica and APG-115ASD powder were placed in a mixer for mixing (mixing speed was 18 rpm, mixing time was 5 minutes). The mixed material was sieved through 40 meshes, and then 21 g of sieved magnesium stearate was added for mixing (the mixing speed was 18 rpm and the mixing time was 5 minutes). The obtained material was placed in a dry granulator for dry granulation, and the obtained particles were collected (roller pressure: 4.0˜7.0 MPa, roller clearance: 1.0˜5.0 mm, roller speed: 3.0˜7.0 rpm, speed of granulating parts: 30˜90 rpm, feed shaft speed: 18 rpm, fine screen: 0.8 mm). 112 g of colloidal silica after screening and 35 g of magnesium stearate after screening were mixed and lubricated with the above particles in turn, and then the materials were pressed with a circular die with a diameter of 10 mm (the feed rotation speed was 5˜30 rpm, the tablet pressing rotation speed was 20˜30 rpm, the tablet thickness scale was 0.3˜5.0 mm, the feed scale was 10.5˜15.5 mm, and the main pressure range was 10˜25 KN), 7000 plain tablets were obtained. 84 g OPADRY® II film coating premix coated plain tablets to obtain coated tablets.

Two batches of coated tablets were produced according to the above process, and their dissolution, content and related substances were determined.

(1) Dissolution

Adopt the method II (slurry method) of dissolution determination in Chinese Pharmacopoeia, 900 ml pH 6.8 phosphate solution (0.2% sodium dodecyl sulfate) was took as the dissolution medium, the rotating speed was 75 revolutions per minute, and the cumulative dissolution amount after 30 minutes was not less than 80% of the marked amount.

TABLE 19

| | Drug dissolution (%) |
|---|---|
| No. | (30 min, 75 rpm) |
| 1 | 95 |
| 2 | 96 |

(2) Content and Related Substances

TABLE 20

| | | | | Content |
|---|---|---|---|---|
| | | | | uniformity |
| | | | | Should be |
| | | Total | | complied with |
| No. | Single impurity | impurities | Content | regulations |
| 1 | Maximum | 0.4% | 96.9% | Consistant |
| | single impurity | | | with the |
| | 0.15% | | | regulations |
| 2 | Maximum | 0.4% | 98.1% | Consistant |
| | single impurity | | | with the |
| | 0.12% | | | regulations |

Effect Example 1

Sample to be Tested:

1. APG-115 particles: 12.5% of APG-115 hydrate, 25.1% of hypromellose acetate succinate, 21.8% of microcrystalline cellulose KG 802, 21.8% of anhydrous calcium phosphate, 4.9% of hydroxypropyl cellulose, 3.2% of croscarmellose sodium, 5.8% of colloidal silica, 1.9% of magnesium stearate and film coating premix (the percentage was mass percentage) were used as raw materials, APG-115 particles were prepared according to the preparation method of example 37;

2. APG-115 Hydrate

Sample Solution Preparation:

After fully grinding and mixing the samples with PEG400 (the concentration in the sample solution was 5%), a small amount (about 2 ml) of 0.2% HPMC aqueous solution was added to grind into a uniform paste until there were no large particles. Under the condition of continuous grinding, 0.2% HPMC aqueous solution was added to a sufficient amount and maintained stirring (800 RPM) for 5 minutes. After sealing the sample solution container with sealing film, it was placed in the ultrasonic instrument (40 kHz) for ultrasonic for 20 minutes (to avoid the rise of ultrasonic medium water temperature during ultrasonic process, the water temperature was controlled within 40° C.); Stopped the ultrasound, mixed the suspension evenly before use (stirring for about 3 minutes), and then sampled immediately (the concentration of the sample was 6 mg/ml).

Test method: three male beagle dogs were selected and divided into test article 1 (APG-115 particles) group and test article 2 (APG-115 hydrate) group. Each group received single oral capsule administration: after the administration of test article 1, after an 8-day cleaning period, the group name was changed to test article 2, and so on. Did not reassign animal numbers.

Blood samples were collected and plasma was separated at 0.5, 1.0, 2.0, 4.0, 6.0, 10, 24, 48, 72, 96, 120, 144 and 168 hours before and after each round of administration. All plasma samples were mailed to the client for testing. The concentration of APG-115 in dog plasma was analyzed by LC-MS/Ms. The lower limit of quantification of the method was 50 ng/ml. The non compartmental model method (NCA) of the metabolic kinetic data analysis software winnonlin 8.0.0.3176 was used to analyze the plasma concentration data and calculate the pharmacokinetic parameters. See table 21 to evaluate the kinetic characteristics of APG-115 in animals after administration.

TABLE 21
pharmacokinetic parameters of APG-115 sample dogs

| | | t _{1/2} | T ₘₐₓ | C ₘₐₓ | AUC ₗₐₛₜ | AUC _{inf} | Vd | CL | MRT |
|---|---|---|---|---|---|---|---|---|---|
| Sample | | (h) | (h) | (ng/ml) | (h · ug/mL) | (h · ug/mL) | (mL/kg) | (mL/h/kg) | (h) |
| APG-115 | Mean | 16.84 | 2.00 | 14833.33 | 372.26 | 374.07 | 139.11 | 5.60 | 23.70 |
| particles | SD | 2.44 | 0.00 | 3061.59 | 102.20 | 101.98 | 50.60 | 1.41 | 3.07 |
| | CV% | 14.48 | 0.00 | 20.64 | 27.45 | 27.26 | 36.37 | 25.18 | 12.94 |
| APG-115 | Mean | 346.43 | 6.00 | 135.67 | 4.58 | 33.42 | 29088.77 | 60.80 | 31.43 |
| hydrate | SD | 167.44 | 3.46 | 30.24 | 1.63 | 5.91 | 9310.27 | 10.76 | 14.62 |
| | CV% | 48.33 | 57.74 | 22.29 | 35.48 | 17.69 | 32.01 | 17.69 | 46.52 |

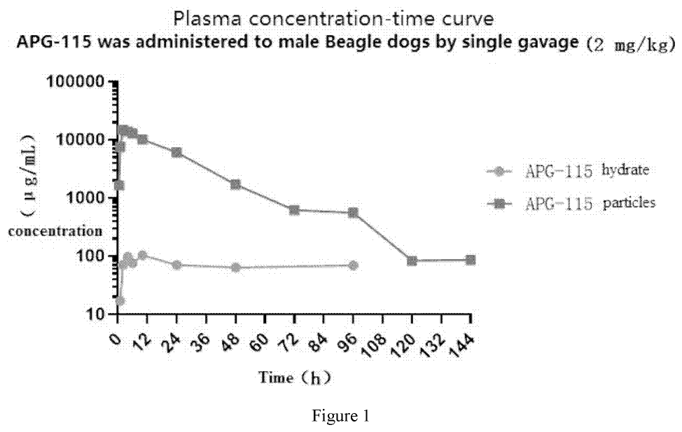

Conclusion: as shown in FIG. 1, the exposure of APG-115 particles was more than ten times that of APG-115 hydrate, and the exposure of APG-115 hydrate in dogs was very low.

Although the specific embodiments of the invention have been described above, those skilled in the art should understand that this is only an example, and the protection scope of the invention is limited by the appended claims. Those skilled in the art can make a variety of changes or modifications to these embodiments without departing from the principle and essence of the invention, but these changes and modifications fall within the protection scope of the invention.

1. A solid dispersion comprising a carrier and an active ingredient of formula (I), or a pharmaceutically acceptable salt, crystal form, or hydrate thereof;

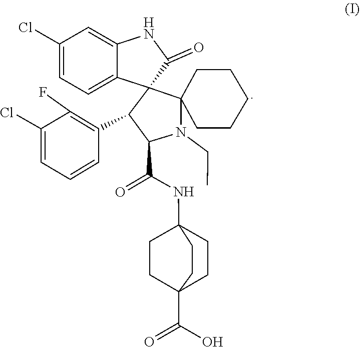 [SMILES: CCN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2]

2. The solid dispersion according to claim 1, wherein the carrier is selected from one or more of homopolymer and copolymer cellulose ester of N-vinyl lactam, pH dependent cellulose derivative, non-ionic water-soluble cellulose ether, cellulose ether, high molecular weight polycyclic oxide, N-vinyl amide polymer, polyacrylate, polymethacrylate, polyacrylamide, vinyl acetate polymer, polyethylene glycol, polyvinyl caprolactam/polyvinyl acetate graft copolymer, oligosaccharide and polysaccharide, and can also be one or more of povidone, covidone, hypromellose acetate succinate, polyethylene glycol/polyvinyl caprolactam/polyvinyl acetate graft copolymer, and can also be one or more of hypromellose acetate succinate, hydroxypropyl cellulose, povidone and acrylic resin;
wherein the pharmaceutically acceptable salt is selected from hydrochloride, hydrobromate, hydroiodate, sulfate, bisulfate, 2-hydroxyethanesulfonate, phosphate, hydrogen phosphate, acetate, adipate, alginate, lysine, arginine, histidine, aspartate, benzoate, bisulfate, butyrate, camphorate, camphor sulfonate, diglucosate, glycerophosphate, hemisulfate, heptanate, caproate, formate, succinate, fumarate, maleate, ascorbate, hydroxyethyl sulfonate, salicylate, methanesulfonate, mesitylene sulfonate, naphthalene sulfonate, nicotinic acid, 2-naphthalene sulfonate, oxalate, dihydroxynaphthalate, pectinate, persulfate, 3-phenylpropionate, picrate, tervalerate, propionate, trichloroacetate, trifluoroacetate, phosphate, glutamate, bicarbonate, p-toluenesulfonate, undecanoate, lactate, citrate, tartrate, gluconate, methanesulfonate, ethyldisulfonate, benzene sulfonate, L-tartrate, maleate, sodium salt, potassium salt, Choline salt, aminobutanol salt, calcium salt or p-toluenesulfonate, or phosphate, sulfate, L-tartrate, hydrochloride, maleate, hydrobromate, methanesulfonate, lysine salt, arginine salt, histidine salt, sodium salt, potassium salt, choline salt, aminobutanol salt and calcium salt;
wherein the hydrate is selected from hemihydrate, monohydrate, dihydrate, trihydrate, tetrahydrate, pentahydrate, hexahydrate, heptahydrate, octahydrate, nonahydrate, decahydrate, undecahydrate and dodecahydrate;
wherein the solid dispersion further comprises an optional antisticking agent selected from one or more of colloidal silica, talc powder, starch, D-leucine, L-leucine, sodium lauryl sulfate and metal stearate; wherein the mass ratio of the antisticking agent to the active ingredient is 0.05:1˜0.08:1.

3. The solid dispersion according to claim 2, wherein,
by mass of 1 part of the active ingredient, the solid dispersion comprises 1 part of the active ingredient and 1.2˜8 parts of the carrier;
or, by mass of 1 part of the active ingredient, the solid dispersion comprises 1 part of the active ingredient and 2˜4 parts of the carrier,
or, by mass of 1 part of the active ingredient, the solid dispersion comprises 1 part of the active ingredient and 1.2˜8 parts of hypromellose acetate succinate;
or, by mass of 1 part of the active ingredient, the solid dispersion comprises 1 part of the active ingredient and 2˜4 parts of hypromellose acetate succinate;
or, by mass of 1 part of the active ingredient, the solid dispersion comprises 1 part of the active ingredient and 2 parts of hypromellose acetate succinate;
or, by mass of 1 part of the active ingredient, the solid dispersion may include 1 part of the active ingredient and 1.2˜8 parts of hydroxypropyl cellulose;
or, by mass of 1 part of the active ingredient, the solid dispersion may include 1 part of the active ingredient and 2˜4 parts of hydroxypropyl cellulose;
or, by mass of 1 part of the active ingredient, the solid dispersion comprises 1 part of the active ingredient and 2 parts of hydroxypropyl cellulose;
or, by mass of 1 part of the active ingredient, the solid dispersion may include 1 part of the active ingredient and 1.2˜8 parts of povidone;
or, by mass of 1 part of the active ingredient, the solid dispersion may include 1 part of the active ingredient and 2˜4 parts of povidone;
or, by mass of 1 part of the active ingredient, the solid dispersion comprises 1 part of the active ingredient and 2 parts of povidone;
or, by mass of 1 part of the active ingredient, the solid dispersion may include 1 part of the active ingredient and 1.2˜8 parts of acrylic resin;
or, by mass of 1 part of the active ingredient, the solid dispersion may include 1 part of the active ingredient and 2˜4 parts of acrylic resin;
or, by mass of 1 part of the active ingredient, the solid dispersion comprises 1 part of the active ingredient and 2 parts of acrylic resin;
or, by mass of 1 part of the active ingredient, the solid dispersion comprises 1 part of the active ingredient, 1.2˜8 parts of the carrier and 0.05˜0.08 parts of the antisticking agent;
or, by mass of 1 part of the active ingredient, the solid dispersion comprises 1 part of the active ingredient, 2˜8 parts of hypromellose acetate succinate and 0.05˜0.08 parts of colloidal silica;
or, by mass of 1 part of the active ingredient, the solid dispersion comprises 1 part of the active ingredient, 2˜4 parts of hypromellose acetate succinate and 0.06˜0.07 parts of colloidal silica;
or, by mass of 1 part of the active ingredient, the solid dispersion includes 1 part of the active ingredient, 2 parts of hypromellose acetate succinate and 0.064 parts of colloidal silica.

4. A method for preparing solid dispersion of claim 2, which comprises the following steps: 1) mixing one or more carrier and the active ingredient with a solvent to obtain a mixed liquid, 2) drying the mixed liquid; and 3) optionally, mixing the mixture with the antisticking agent before drying.

5. The method for preparing the solid dispersion according to claim 4, wherein,
The mass volume ratio of the active ingredient to the solvent is 5:1˜30:1 mg/ml, or 15:1˜25:1 mg/ml;
wherein the solvent is one or more of alcohol solvent, water, ester solvent, ketone solvent, halogenated hydrocarbon solvent, nitrile solvent and ether solvent, which can be ester solvent, ether solvent or ester solvent;
wherein the alcohol solvent can be ethanol; the ester solvent can be methyl acetate; the ether solvent can be tetrahydrofuran; the ketone solvent can be acetone; the halogenated hydrocarbon solvent can be dichloromethane; the nitrile solvent can be acetonitrile;
wherein the drying includes a first drying and a second drying; wherein the first drying can be spray drying or fluidized bed boiling drying; the second drying can be vacuum decompression drying or electric blast drying.

6. A pharmaceutical composition comprising a solid dispersion according to claim 2 and excipients.

7. The pharmaceutical composition according to claim 6, wherein,
the excipient comprises one or more of an absorption enhancer, an antioxidant, a dry antisticking agent, a buffer, a coating material, a coating material, a diluent, a disintegrating agent, an emulsifier, a flavor agent, a humectant, a lubricant, an antisticking agent, a glidant, a preservative, a solubilizer, a corrective and a releasing agent;
wherein the diluent may include one or more of cellulose, lactose, lactose alcohol, maltitol, mannitol, sorbitol, xylitol, glucose, fructose, sucrose and sucrose based diluent, maltose, inositol, hydrolyzed grain solid, starch, starch component, dextrin, calcium salt, magnesium salt, bentonite, kaolin and sodium chloride; wherein the diluent can be one or more of microcrystalline cellulose, silicified microcrystalline cellulose, calcium phosphate, pregelatinized starch, lactose, mannitol and anhydrous calcium hydrogen phosphate, or one or more of microcrystalline cellulose, silicified microcrystalline cellulose, pregelatinized starch, calcium phosphate and anhydrous calcium hydrogen phosphate, or one or more of microcrystalline cellulose, pregelatinized starch and calcium phosphate, or a mixture of microcrystalline cellulose and anhydrous calcium hydrogen phosphate; wherein the microcrystalline cellulose can be PH 102 and/or KG 802; when the diluent is a mixture of microcrystalline cellulose and anhydrous calcium hydrogen phosphate, the mass ratio of microcrystalline cellulose to anhydrous calcium hydrogen phosphate can be 0.1:1˜10:1, or 0.5:1˜2:1;
wherein the disintegrating agent may include one or more of starch, clay, magnesium aluminum silicate, cellulose based disintegrating agent, alginate, povidone, crospovidone, polacrine potassium, gum and colloidal silica; The disintegrating agent can be croscarmellose sodium and/or crospovidone;
wherein the lubricant may include one or more of glyceryl behenate, stearic acid and its salts, hydrogenated vegetable oil, glyceryl palmitate, talc powder, wax, sodium benzoate, sodium acetate, sodium fumarate, sodium stearate fumarate, PEG, poloxamer, polyvinyl alcohol, sodium oleate, sodium lauryl sulfate and magnesium lauryl sulfate; wherein lubricant can be magnesium stearate.

8. The pharmaceutical composition of claim 7 wherein the pharmaceutical composition optionally includes an antisticking agent and a glidant; wherein the pharmaceutical composition optionally further comprise a dry antisticking agent;
wherein the mass ratio of the diluent to the solid dispersion can be 0.2:1˜8:1, 0.5:1˜8:1, 0.8:1˜2:1 or 0.5:1˜1:1;
wherein the mass ratio of the disintegrating agent to the solid dispersion can be 0.03:1˜0.3:1, 0.1:1˜0.2:1, 0.05:1˜0.2:1 or 0.05:1˜0.15:1;
wherein the mass ratio of the lubricant to the solid dispersion can be 0.005:1˜0.2:1, 0.01:1˜0.2:1, 0.02:1˜0.04:1 or 0.01:1˜0.02:1;
wherein the antisticking agent may include one or more of talc powder, colloidal silica, starch, D-leucine, L-leucine, sodium lauryl sulfate and metal stearate;
wherein the antisticking agent can be colloidal silica;
wherein the glidant may include one or more of colloidal silica, starch, powdered cellulose, sodium lauryl sulfate, magnesium trisilicate and metal stearate;
wherein the glidant can be colloidal silica;
wherein the mass ratio of the total mass of the antisticking agent and the glidant to the mass of the solid dispersion can be 0.02:1˜0.3:1, 0.05:1˜0.1:1 or 0.1:1˜0.2:1;
wherein the dry antisticking agent may include one or more of gum Arabic, tragacanth gum, glucose, polyglucose, starch, gelatin, modified cellulose, dextrin, zein, alginic acid and alginate, magnesium aluminum silicate, bentonite, polyethylene glycol, polyethylene oxide, guar gum, polysaccharide acid, polyvinylpyrrolidone, polyacrylic acid, polymethacrylate, etc.;
wherein the dry antisticking agent can be hydroxypropyl cellulose;
wherein the mass ratio of the dry antisticking agent to the solid dispersion is 0.02:1˜0.5:1, which can be 0.1:1˜0.3:1.

9. The pharmaceutical composition according to claim 7, wherein,
by mass of 1 part of the solid dispersion, the pharmaceutical composition comprises 1 part of the solid dispersion, 0.2˜8 parts of the diluent, 0.03˜0.3 parts of the disintegrating agent and 0.005˜0.2 parts of the lubricant;
or, by mass of 1 part of the solid dispersion, the pharmaceutical composition comprises 1 part of solid dispersion, 0.2˜8 parts of diluent, 0.02˜0.3 parts of the antisticking agent and glidant, 0.03˜0.3 parts of disintegrating agent and 0.005˜0.2 parts of lubricant;
or, by mass of 1 part of the solid dispersion, the pharmaceutical composition comprises 1 part of the solid dispersion, 0.2˜8 parts of the diluent, 0.02˜0.5 parts of the dry antisticking agent, 0.02˜0.3 parts of the antisticking agent and glidant, 0.03˜0.3 parts of the disintegrating agent and 0.005˜0.2 parts of the lubricant.

10. The pharmaceutical composition according to claim 7, wherein,
by mass of 1 part of the solid dispersion, the pharmaceutical composition comprises 1 part of the solid dispersion, 0.5˜1 part of silicified microcrystalline cellulose, 0.1˜0.2 part of crospovidone and 0.01˜0.02 part of magnesium stearate;
or, by mass of 1 part of the solid dispersion, the pharmaceutical composition comprises 1 part of the solid dispersion, 0.65˜0.85 parts of silicified microcrystalline cellulose, 0.13˜0.17 parts of crospovidone and 0.012˜0.016 parts of magnesium stearate;
or, by mass of 1 part of the solid dispersion, the pharmaceutical composition comprises 1 part of the solid dispersion, 0.8˜2 parts of microcrystalline cellulose PH 102, 0.05˜0.2 parts of croscarmellose sodium, 0.05˜0.1 parts of colloidal silica and 0.02˜0.04 parts of magnesium stearate;
or, by mass of 1 part of the solid dispersion, the pharmaceutical composition comprises 1 part of the solid dispersion, 1.26˜1.46 parts of microcrystalline cellulose PH 102, 0.11˜0.15 parts of croscarmellose sodium, 0.06˜0.1 parts of colloidal silica and 0.02˜0.04 parts of magnesium stearate;
or, by mass of 1 part of the solid dispersion, the pharmaceutical composition comprises 1 part of the solid dispersion, 0.53˜1.32 parts of microcrystalline cellulose PH 102, 0.27˜0.68 parts of pregelatinized starch, 0.05˜0.2 parts of croscarmellose sodium, 0.05˜0.1 parts of colloidal silica and 0.02˜0.04 parts of magnesium stearate;
or, by mass of 1 part of the solid dispersion, the pharmaceutical composition comprises 1 part of the solid dispersion, 0.81˜1.01 part of microcrystalline cellulose PH 102, 0.35˜0.55 part of pregelatinized starch, 0.11˜0.15 part of croscarmellose sodium, 0.06˜0.1 part of colloidal silica and 0.02˜0.04 part of magnesium stearate;
or, by mass of 1 part of the solid dispersion, the pharmaceutical composition comprises 1 part of the solid dispersion, 0.4˜1 part of microcrystalline cellulose PH 102, 0.4˜1 part of calcium phosphate, 0.05˜0.2 part of croscarmellose sodium, 0.05˜0.1 part of colloidal silica and 0.02˜0.04 part of magnesium stearate;
or, by mass of 1 part of the solid dispersion, the pharmaceutical composition comprises 1 part of the solid dispersion, 0.58˜0.78 part of microcrystalline cellulose PH 102, 0.58˜0.78 part of calcium phosphate, 0.11˜0.15 part of croscarmellose sodium, 0.07˜0.09 part of colloidal silica and 0.02˜0.04 part of magnesium stearate;
or, by mass of 1 part of the solid dispersion, the pharmaceutical composition comprises 1 part of the solid dispersion, 0.4˜1 part of microcrystalline cellulose KG 802, 0.4˜1 part of anhydrous calcium phosphate, 0.05˜0.2 part of croscarmellose sodium, 0.05˜0.1 part of colloidal silica and 0.02˜0.04 part of magnesium stearate;
or, by mass of 1 part of the solid dispersion, the pharmaceutical composition comprises 1 part of the solid dispersion, 0.61˜0.81 parts of microcrystalline cellulose KG 802, 0.61˜0.81 parts of anhydrous calcium phosphate, 0.068˜0.088 parts of croscarmellose sodium, 0.05˜0.054 parts of colloidal silicon dioxide and 0.03˜0.04 parts of magnesium stearate;
or, by mass of 1 part of the solid dispersion, the pharmaceutical composition comprises 1 part of the solid dispersion, 0.25˜4 parts of microcrystalline cellulose KG 802, 0.25˜4 parts of calcium phosphate, 0.03˜0.3 parts of croscarmellose sodium, 0.02˜0.5 parts of hydroxypropyl cellulose, 0.02˜0.3 parts of colloidal silica and 0.02˜0.3 parts of magnesium stearate;
or, by mass of 1 part of the solid dispersion, the pharmaceutical composition comprises 1 part of the solid dispersion, 0.5˜0.7 parts of microcrystalline cellulose KG 802, 0.5˜0.7 parts of calcium phosphate, 0.11˜0.15 parts of croscarmellose sodium, 0.14˜0.18 parts of hydroxypropyl cellulose, 0.04˜0.08 parts of colloidal silica and 0.02˜0.04 parts of magnesium stearate;
or, by mass of 1 part of the solid dispersion, the pharmaceutical composition comprises 1 part of the solid dispersion, 0.25˜4 parts of microcrystalline cellulose KG 802, 0.25˜4 parts of anhydrous calcium phosphate, 0.03˜0.3 parts of croscarmellose sodium, 0.02˜0.5 parts of hydroxypropyl cellulose, 0.02˜0.3 parts of colloidal silica and 0.02˜0.3 parts of magnesium stearate;
or, by mass of 1 part of the solid dispersion, the pharmaceutical composition comprises 1 part of the solid dispersion, 0.55˜0.75 parts of microcrystalline cellulose KG 802, 0.55˜0.75 parts of anhydrous calcium phosphate, 0.07˜0.15 parts of croscarmellose sodium, 0.07˜0.18 parts of hydroxypropyl cellulose, 0.04˜0.15 parts of colloidal silica and 0.02˜0.06 pans of magnesium stearate.

11. The pharmaceutical preparation comprising the pharmaceutical composition according to claim 7; wherein the pharmaceutical preparation can be a solid preparation or a powder, granule, tablet, capsule, dropping pill, or film.

12. A method for preparing tablets, comprising the following steps:
Step 1: mixing a sold dispersion of claim 2, a diluent, and a disintegrating agent to obtain a mixture: sieving and granulating the mixture to obtain particles, optionally, an antisticking agent or a glidant is added to the mixture before sieving: optionally a lubricant is added after sieving before granulating;
Step 2): mixing the particles of step 1 with the lubricant and pressing the particles to obtain a tablet; optionally, the particles of step 1 are mixed with the antisticking agent and glidant.

13. The preparation method of the tablet according to claim 12, wherein,
when part of the antisticking agent and glidant is added in step 1, and the remaining part of the antisticking agent and glidant is added in step 2, the total amount of the antisticking agent and glidant is 100%, and the mass percentage of the antisticking agent and glidant in step 1 is 0.5%˜20%;
wherein when part of the lubricant is added in step 1 and the remaining part of the lubricant is added in step 2, the total amount of the lubricant is 100%, and the mass percentage of the lubricant in step 1 is 0.1%-10%;
where in step 1, the granulation is dry granulation.

14. A coated tablet comprising the pharmaceutical composition according to claim 7.

15. The coated tablet according to claim 14, wherein,
the mass ratio of the pharmaceutical composition and the coating in the coated tablet is 0.02:1˜0.2:1, which can be 0.05:1˜0.1:1;
wherein the coating in the coated tablet includes polyvinyl alcohol, titanium dioxide, talc powder, triethylglycerol and hydroxypropyl methyl cellulose, which can be a film coating premix.

16. A method for the treatment of diseases caused by P53 and/or MDM2 abnormalities, wherein diseases are cancer or hyperproliferative diseases.

17. The method according to claim 16, wherein cancer is adrenal cortical cancer, advanced cancer, anal cancer, aplastic anemia, cholangiocarcinoma, bladder cancer, bone cancer, bone metastasis, adult brain/CNS tumor, childhood brain/CNS tumor, breast cancer, male breast cancer, childhood cancer, unknown primary cancer, giant lymph node hyperplasia, cervical cancer, colorectal/rectal cancer, endometrial cancer, esophageal cancer, Ewing's tumor family, eye cancer, gallbladder cancer, gastrointestinal carcinoid tumor, gastrointestinal stromal tumor, gestational trophoblastic disease, Hodgkin's disease, Kaposi's sarcoma, renal cancer, laryngeal and hypopharyngeal cancer, adult acute lymphocytic leukemia, acute myeloid leukemia, chronic lymphocytic leukemia, chronic myeloid leukemia, chronic myelomonocytic leukemia, childhood leukemia, liver cancer, non-small cell lung cancer, small cell lung cancer, lung carcinoid tumor, skin lymphoma, malignant mesothelioma, multiple myeloma, myelodysplastic syndrome, nasal cavity and paranasal sinus cancer, nasopharyngeal carcinoma, neuroblastoma, non-Hodgkin's lymphoma, childhood non-Hodgkin's lymphoma, oral and oropharyngeal carcinoma, osteosarcoma, ovarian cancer, pancreatic cancer, penile cancer, pituitary adenocarcinoma, prostate cancer, retinoblastoma, rhabdomyosarcoma, salivary gland cancer, sarcoma—adult soft tissue carcinoma, basal skin cancer and squamous cell carcinoma, skin cancer melanoma, small bowel cancer, gastric cancer, testicular cancer, thymic carcinoma, thyroid cancer, uterine sarcoma, vaginal cancer, vulvar cancer, Waldenstrom's macroglobulinemia, or Wilms tumor.

18. The method of claim 12, wherein the diluent is cellulose, lactose, lactose alcohol, maltitol, mannitol, sorbitol, xylitol, glucose, fructose, sucrose and sucrose based diluent, maltose, inositol, hydrolyzed grain solid, starch, starch component, dextrin, calcium salt, magnesium salt, bentonite, kaolin and sodium chloride, microcrystalline cellulose, silicified microcrystalline cellulose, calcium phosphate, pregelatinized starch, lactose, mannitol and anhydrous calcium hydrogen phosphate, calcium phosphate, or anhydrous calcium hydrogen phosphate, or a mixture of microcrystalline cellulose and anhydrous calcium hydrogen phosphate; and wherein the disintegrating is starch, clay, magnesium aluminum silicate, cellulose based disintegrating agent, alginate, povidone, crospovidone, polacrine potassium, gum, or colloidal silica.

19. The method of claim 12, wherein the antisticking agent is colloidal silica, talc powder, starch, D-leucine, L-leucine, sodium lauryl sulfate or metal stearate; and wherein the glidant is colloidal silica, starch, powdered cellulose, sodium lauryl sulfate, magnesium trisilicate or metal stearate.

20. The method of claim 19, wherein the microcrystalline cellulose is PH 102 or KG 802; wherein the disintegrating agent is croscarmellose sodium or crospovidone.
